(19) United States
(12) Patent Application Publication
(10) Pub. No.: US 2025/0197516 A1
(43) Pub. Date: Jun. 19, 2025

(54) HIGH AFFINITY HUMAN ANTIBODIES TO HUMAN IL-4 RECEPTOR

(71) Applicant: REGENERON PHARMACEUTICALS, INC., Tarrytown, NY (US)

(72) Inventors: Joel H. MARTIN, Putnam Valley, NY (US); Tammy T. HUANG, Cross River, NY (US); Jeanette L. FAIRHURST, White Plains, NY (US); Nicholas J. PAPADOPOULOS, LaGrangeville, NY (US); George D. YANCOPOULOS, Yorktown Heights, NY (US)

(21) Appl. No.: 18/935,211

(22) Filed: Nov. 1, 2024

Related U.S. Application Data
(63) Continuation of application No. 17/064,393, filed on Oct. 6, 2020, now Pat. No. 12,162,943.
Application No. 17/064,393 is a continuation of application No. 15/874,635, filed on Jan. 18, 2018, now abandoned.
Application No. 15/874,635 is a continuation of application No. 14/254,369, filed on Apr. 16, 2014, now abandoned.
Application No. 14/254,369 is a continuation of application No. 13/678,650, filed on Nov. 16, 2012, now Pat. No. 8,735,095.
Application No. 13/678,650 is a division of application No. 13/287,151, filed on Nov. 2, 2011, now Pat. No. 8,337,839.
Application No. 13/287,151 is a division of application No. 12/556,605, filed on Sep. 10, 2009, now Pat. No. 8,075,887.
Application No. 12/556,605 is a division of application No. 12/260,307, filed on Oct. 29, 2008, now Pat. No. 7,608,693.
Application No. 12/260,307 is a continuation-in-part of application No. 11/906,517, filed on Oct. 2, 2007, now Pat. No. 7,605,237.
(60) Provisional application No. 60/957,738, filed on Aug. 24, 2007.
(60) Provisional application No. 60/848,694, filed on Oct. 2, 2006.

(51) Int. Cl. C07K 16/28; A61K 39/00; A61K 39/395; A61K 45/06

(52) U.S. Cl. CPC C07K 16/2866; A61K 39/3955; A61K 45/06; A61K 2039/505; C07K 2317/21; C07K 2317/76; C07K 2317/92

(57) ABSTRACT

The present invention provides nucleic acid molecules that encode antibodies or antigen-binding fragments thereof, which specifically bind human interleukin-4 receptor (IL-4R). Also provided are expression vectors comprising nucleic acid molecule that encode anti-IL-4R antibodies, host cells comprising the expression vectors, and methods of producing anti-IL-4R antibodies or antigen-binding fragments thereof comprising growing the host cells under conditions permitting production of the antibody or fragment, and recovering the antibody or fragment so produced.

### CROSS-REFERENCE TO RELATED APPLICATIONS

This application is a continuation of U.S. patent application Ser. No. 17/064,393, filed on Oct. 6, 2020, which is a continuation of U.S. patent application Ser. No. 15/874,635, filed on Jan. 18, 2018, now abandoned, which is a continuation of U.S. patent application Ser. No. 14/254,369, filed on Apr. 16, 2014, now abandoned, which is a continuation of U.S. patent application Ser. No. 13/678,650, filed on Nov. 16, 2012, issued as U.S. Pat. No. 8,735,095 on May 27, 2014, which is a divisional of U.S. patent application Ser. No. 13/287,151, filed on Nov. 2, 2011, issued as U.S. Pat. No. 8,337,839 on Dec. 25, 2012, which is a divisional of U.S. patent application Ser. No. 12/556,605, filed on Sep. 10, 2009, issued as U.S. Pat. No. 8,075,887 on Dec. 13, 2011, which is a divisional of U.S. patent application Ser. No. 12/260,307, filed on Oct. 29, 2008, issued as U.S. Pat. No. 7,608,693 on Oct. 27, 2009, which is a continuation-in-part of U.S. patent application Ser. No. 11/906,517, filed on Oct. 2, 2007, issued as U.S. Pat. No. 7,605,237 on Oct. 20, 2009, which claims the benefit under 35 USC § 119 (e) of U.S. Provisional Application No. 60/957,738, filed on Aug. 24, 2007, and U.S. Provisional Application No. 60/848,694, filed on Oct. 2, 2006, the contents of which are herein specifically incorporated by reference in their entireties.

### REFERENCE TO A SEQUENCE LISTING XML

This application contains a Sequence Listing that has been submitted electronically in XML format. The Sequence Listing XML is incorporated herein by reference. Said XML file, created on Oct. 31, 2024, is named 40848_0089USC4_SL.xml and is 405,504 bytes in size.

### BACKGROUND

Interleukin-4 (IL-4, also known as B cell stimulating factor or BSF-1) was originally characterized by its ability to stimulate the proliferation of B cells in response to low concentrations of antibodies directed to surface immunoglobulin. IL-4 has been shown to possess a broad spectrum of biological activities, including growth stimulation of T cells, mast cells, granulocytes, megakaryocytes and erythrocytes. IL-4 induces the expression of class II major histocompatibility complex molecules in resting B cells, and enhances the secretion of IgE and IgG1 isotypes by stimulated B cells.

The biological activities of IL-4 are mediated by specific cell surface receptors for IL-4. Human IL-4 receptor alpha (hIL-4R) (SEQ ID NO:274) is described in, for example, U.S. Pat. Nos. 5,599,905, 5,767,065, and 5,840,869. Antibodies to hIL-4R are described in U.S. Pat. Nos. 5,717,072 and 7, 186,809.

Methods to produce antibodies useful as human therapeutics include generating chimeric antibodies and humanized antibodies (see, for example, U.S. Pat. No. 6,949,245). See, for example, WO 94/02602 and U.S. Pat. No. 6,596,541 (both of which publications are herein specifically incorporated by reference) describing methods of generating nonhuman transgenic mice capable of producing human antibodies.

Methods for using antibodies to hIL-4R are described in U.S. Pat. Nos. 5,714,146; 5,985,280; and 6,716,587.

### BRIEF SUMMARY OF THE INVENTION

In a first aspect, the invention provides human antibodies, preferably recombinant human antibodies, that specifically bind human interleukin-4 receptor (hIL-4R). The human antibodies are characterized by binding to hIL-4R with high affinity and by the ability to neutralize hIL-4 activity. In specific embodiments, the human antibodies are capable of blocking hIL-13/hIL-13R1 complex binding to hIL-4R, and thus inhibit signaling by hIL-13. The antibodies can be full-length (for example, an IgG1 or IgG4 antibody) or may comprise only an antigen-binding portion (for example, a Fab, F(ab′)₂ or scFv fragment), and may be modified to effect functionality, e.g., to eliminate residual effector functions (Reddy et al. (2000) J. Immunol. 164:1925-1933).

In a general embodiment, the invention provides an antibody or antigen-binding fragment thereof, that specifically binds hIL-4R (SEQ ID NO:274) with a K_{D} of about 300 PM or less, as measured by surface plasmon resonance in a monomeric or dimeric assay. In a more specific embodiment, the antibody or antigen-binding portion thereof exhibits a K_{D} of about 200 pM or less, about 150 or less, about 100 pM or less, or about 50 pM. In various embodiments, the antibody or antigen-binding fragment blocks hIL-4 activity with an IC₅₀ of about 100 pM or less, as measured by luciferase bioassay. In more specific embodiments, the antibody or antigen-binding fragment exhibits an IC₅₀ of about 50 pM or less, about 30 PM or less, or about 25 pM or less, as measured by STAT6 luciferase bioassay. In various embodiments, the antibody or antigen-binding fragment blocks hIL-13 activity with an IC₅₀ of about 100 pM or less, about 90 pM or less, about 50 pM or less, or about 20 pM or less, as measured by STAT6 luciferase bioassay.

In a second aspect, the antibody of the invention comprises a heavy chain variable region (HCVR) sequence selected from the group consisting of SEQ ID NO:2, 18, 22, 26, 42, 46, 50, 66, 70, 74, 90, 94, 98, 114, 118, 122, 138, 142, 146, 162, 166, 170, 186, 190, 194, 210, 214, 218, 234, 238, 242, 258 and 262, or a substantially similar sequence thereof.

In a third aspect, the antibody of the invention comprises a light chain variable region (LCVR) sequence selected from the group consisting of SEQ ID NO:10, 20, 24, 34, 44, 48, 58, 68, 72, 82, 92, 96, 106, 116, 120, 130, 140, 144, 154, 164, 168, 178, 188, 192, 202, 212, 216, 226, 236, 240, 250, 260 and 264, or a substantially similar sequence thereof.

In one embodiment, the antibody or antibody fragment of the invention comprises HCVR and LCVR sequence pairs (HCVR/LCVR) selected from the group consisting of SEQ ID NO: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260 and 262/264. In a preferred embodiment, the antibody or antibody fragment comprise HCVR/LCVR sequence pairs SEQ ID NO: 162/164, 210/212 and 18/20. In an even more preferred embodiment, the antibody or antibody fragment comprises HCVR/LCVR SEQ ID NO: 162/164.

In a fourth aspect, the invention provides nucleic acid molecules encoding an HCVR, wherein the nucleic acid molecule is a nucleotide sequence selected from the group consisting of SEQ ID NO: 1, 17, 21, 25, 41, 45, 49, 65, 69, 73, 89, 93, 97, 113, 117, 121, 137, 141, 145, 161, 165, 169, 185, 189, 193, 209, 213, 217, 233, 237, 241, 257 and 261, or a substantially identical sequence having at least 95% homology thereof.

In a fifth aspect, the invention provides nucleic acid molecules encoding a LCVR, wherein the nucleic acid molecule is a sequence selected from the group consisting of SEQ ID NO: 9, 19, 23, 33, 43, 47, 57, 67, 71, 81, 91, 95, 105, 115, 119, 129, 139, 143, 153, 163, 167, 177, 187, 191, 201, 211, 215, 225, 235, 239, 249, 259 and 263, or a substantially identical sequence having at least 95% homology thereof.

In one embodiment, the antibody of the invention comprises a HCVR and LCVR encoded by a nucleotide sequence pairs selected from the group consisting of SEQ ID NO: 1/9, 17/19, 21/22, 25/33, 41/43, 45/47, 49/57, 65/67, 69/71, 73/81, 89/91, 93/95, 97/105, 113/115, 117/119, 121/129, 137/139, 141/143, 145/153, 161/163, 165/167, 169/177, 185/187, 189/191, 193/201, 209/211, 213/215, 217/225, 233/235, 237/239, 241/249, 257/259 and 261/263. In a preferred embodiment, the antibody or antibody fragment comprise HCVR/LCVR sequences encoded by nucleic acid sequences selected from SEQ ID NO: 161/163, 209/211 and 17/19. In an even more preferred embodiment, the antibody or antibody fragment comprises HCVR/LCVR encoded by nucleic acid sequences SEQ ID NO: 161/163.

In a sixth aspect, the invention provides an antibody or antigen-binding fragment comprising a HCDR3 and a LCDR3, wherein the HCDR3 domain selected from the group consisting of SEQ ID NO:8, 32, 56, 80, 104, 128, 152, 176, 200, 224 and 248; and the LCDR3 domain selected from the group consisting of SEQ ID NO: 16, 40, 64, 88, 112, 136, 160, 184, 208, 232 and 256. In a preferred embodiment, the HCDR3/LCDR3 sequences are SEQ ID NO: 152/160, 8/16 or 200/208. In an even more preferred embodiment, the HCDR3 and LCDR3 sequences are SEQ ID NO: 152 and 160.

In a further embodiment, the antibody or antibody fragment further comprises a HCDR1 sequence selected from the group consisting of SEQ ID NO:4, 28, 52, 76, 100, 124, 148, 172, 196, 220 and 244, or a substantially similar sequence thereof; a HCDR2 sequence selected from the group consisting of SEQ ID NO:6, 30, 54, 78, 102, 126, 150, 174, 198, 222 and 246, or a substantially similar sequence thereof; a HCDR3 sequence selected from the group consisting of SEQ ID NO:8, 32, 56, 80, 104, 128, 152, 176, 200, 224 and 248, or a substantially similar sequence thereof; a LCDR1 sequence selected from the group consisting of SEQ ID NO:12, 36, 60, 84, 108, 132, 156, 180, 204, 228 and 252, or a substantially similar sequence thereof; a LCDR2 sequence selected from the group consisting of SEQ ID NO: 14, 38, 62, 86, 110, 134, 158, 182, 206, 230 and 252, or a substantially similar sequence thereof; and a LCDR3 sequence selected from the group consisting of SEQ ID NO: 16, 40, 64, 88, 112, 136, 160, 184, 208, 232 and 256 or a substantially similar sequences thereof. In a preferred embodiment, the antibody or antigen-binding fragment comprise HCDR sequences SEQ ID NO: 148, 150 and 152 and LCDR sequences SEQ ID NO: 156, 158 and 160; HCDR sequences SEQ ID NO:4, 6 and 8 and LCDR sequences SEQ ID NO: 12, 14 and 16; and HCDR sequences SEQ ID NO: 196, 198 and 200 and LCDR sequences SEQ ID NO:204, 206 and 208.

In a seventh aspect, the invention features a human antibody or antibody fragment comprising a HCDR3 and LCDR3, wherein the HCDR3 is encoded by a nucleotide sequence selected from the group consisting of SEQ ID NO:7, 31, 55, 79, 103, 127, 151, 175, 199, 223 and 247, or a substantially identical sequence having at least 95% homology thereof; and the LCDR3 is encoded by a nucleotide sequence selected from the group consisting of SEQ ID NO: 15, 39, 63, 87, 111, 135, 159, 183, 207, 231 and 255, or a substantially identical sequence having at least 95% homology thereof.

In a further embodiment, the invention features a human antibody or antibody fragment comprising a HCDR1 domain encoded by a nucleotide sequence selected from the group consisting of SEQ ID NO:3, 27, 51, 75, 99, 123, 147, 171, 195, 219 and 243, or a substantially identical sequence having at least 95% homology thereof; a HCDR2 domain encoded by a nucleotide sequence selected from the group consisting of SEQ ID NO:5, 29, 53, 77, 101, 125, 149, 173, 197, 221 and 245, or a substantially identical sequence having at least 95% homology thereof; a HCDR3 domain encoded by a nucleotide sequence selected from the group consisting of SEQ ID NO: 7, 31, 55, 79, 103, 127, 151, 175, 199, 223 and 247, or a substantially similar sequence having at least 95% homology thereof; a LCDR1 domain encoded by a nucleotide sequence selected from the group consisting of SEQ ID NO: 11, 35, 59, 83, 107, 131, 155, 179, 203, 227 and 251, or a substantially similar sequence having at least 95% homology thereof; a LCDR2 domain encoded by a nucleotide sequence selected from the group consisting of SEQ ID NO: 13, 37, 61, 85, 109, 133, 157, 181, 205, 229 and 253, or a substantially similar sequence having at least 95% homology thereof; and LCDR3 domain encoded by a nucleotide sequence selected from the group consisting of SEQ ID NO: 15, 39, 63, 87, 111, 135, 159, 183, 207, 231 and 255, or a substantially similar sequence having at least 95% homology thereof. In a preferred embodiment, the antibody or antigen-binding fragment comprise HCDR and LCDR sequences encoded by nucleotide sequences SEQ ID NO: 147, 149, 151, 155, 157 and 159; 195, 197, 199, 203, 205 and 207; and 3, 5, 7, 11, 13 and 15.

In a specific embodiment, the anti-hIL-4R antibody or antigen-binding fragment of the invention comprises HCVR comprising the amino acid sequence shown in SEQ ID NO: 162 and LCVR comprising the amino acid sequence shown in SEQ ID NO: 164, and is characterized by a K_{D} of about 100 pM or less (monomeric substrate) or 70 pM or less (dimeric substrate); a K_{D} of about 160 pM or less (monomeric substrate) or 40 pM or less (dimeric substrate) at 25° C. and 37° C., respectively; and an IC₅₀ of about 10 pM or less (25 pM dimer substrate) or about 100 pM or less (200 pM monomer substrate), which is capable of blocking both hIL-4 and hIL-13 activity with an IC₅₀ of about 30 PM or less (as measured by bioassay) and cross-reacts with monkey IL-4R.

In a specific embodiment, the anti-hIL-4R antibody or antigen-binding fragment of the invention comprises HCVR comprising the amino acid sequence shown in SEQ ID NO: 18 and LCVR comprising the amino acid sequence shown in SEQ ID NO:20, and is characterized by a K_{D} of about 450 pM or less (monomeric or dimeric substrate); and an IC₅₀ of about 40 pM or less (25 pM dimer substrate) or about 100 pM or less (200 pM monomer substrate), which is capable of blocking both hIL-4 and hIL-13 activity with an IC₅₀ of about 100 pM or less (as measured by bioassay).

In a specific embodiment, the anti-hIL-4R antibody or antigen-binding fragment of the invention comprises HCVR comprising the amino acid sequence shown in SEQ ID NO:210 and LCVR comprising the amino acid sequence shown in SEQ ID NO:212, and is characterized by a K_{D} of about 50 pM or less (monomeric substrate) or 30 PM or less (dimeric substrate); a K_{D} of about 200 pM or less (monomeric substrate) or 40 pM or less (dimeric substrate) at 25° C. and 37° C., respectively; and an IC₅₀ of about 10 pM or less (25 pM dimer substrate) or about 90 pM or less (200 pM monomer substrate), which is capable of blocking both hIL-4 and hIL-13 activity with an IC₅₀ of about 25 pM or less (as measured by bioassay) and does not cross-reacts with monkey IL-4R.

In a eighth aspect, the invention features an antibody or antigen-binding fragment of an antibody that specifically binds hIL-4R, comprising three heavy chain and three light chain complementarity determining regions (HCDR1, HCDR2, HCDR3, LCDR1, LCDR2, LCDR3), wherein the HCDR1 comprises an amino acid sequence of the formula X¹-X²-X³-X₄-X⁵-X⁶-X⁷-X⁸ (SEQ ID NO:265), wherein X¹=Gly; X²=Phe; X³=Thr; X⁴=Phe; X⁵=Asp or Arg; X⁶=Asp or Ser; X⁷=Tyr; and X⁸=Ala or Gly; the HCDR2 comprises an amino acid sequence of the formula X¹-X²-X³-X₄-X⁵-X⁶-X⁷-X⁸ (SEQ ID NO:266), wherein X¹=Ile or Leu, X²=Ser, X³=Gly, Tyr or Arg, X⁴=Ser, Asp or Thr, X⁵=Gly or Ser, X⁶=Gly, Ser or Val, X⁷=Ser or Asn, and X⁸=Thr, Lys or Ile; the HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴_ X¹⁵-X¹⁶_ X¹⁷-X¹⁸ (SEQ ID NO:267) wherein X¹=Ala, X²=Lys, X³=Asp, Glu or Trp, X⁴=Gly or Arg, X⁵=Leu, Thr or Arg, X⁶=Gly, Arg or Ser, X⁷=Ile or Gly, X⁸=Thr, Phe or Tyr, X⁹=Ile, Asp or Phe, X¹⁰=Arg, Tyr or Asp, X¹¹=Pro, Tyr or absent, X¹²=Arg or absent, X¹³=Tyr or absent, X¹⁴=Tyr or absent, X¹⁵=Gly or absent, X¹⁶=Leu or absent, X¹⁷=Asp or absent, and X¹⁸=Val or absent; the LCDR1 comprises an amino acid sequence of the formula X¹-X²-X³-X₄-X⁵-X⁶-X⁷-X⁸-X⁹-X¹⁰-X¹¹ (SEQ ID NO: 268) wherein X¹=Gln, X²=Asp, Ser or Val, X³=Ile or Leu, X⁴=Ser, Leu or Asn, X⁵=Asn, Tyr or Ile, X⁶=Trp, Ser or Tyr; X⁷=Ile or absent; X⁸=Gly or absent; X⁹=Tyr or absent; X¹⁰=Asn or absent; and X¹¹=Tyr or absent; the LCDR2 comprises an amino acid sequence of the formula X¹-X²-X³ (SEQ ID NO:269) wherein X¹=Leu, Ala or Val, X²=Ala or Gly, and X³=Ser; and the LCDR3 comprises an amino acid sequence of the formula X¹-X²-X³-X⁴-X⁵-X⁶-X⁷-X⁸-X⁹ (SEQ ID NO:270) wherein X¹=Gln or Met, X²=Gln, X³=Ala or Tyr, X⁴=Leu or Asn, X⁵=Gln or Ser, X⁶=Thr, Phe or His, X⁷=Pro, X⁸=Tyr, Ile or Trp, and X⁹=Thr.

In a more specific embodiment, the HCDR1 comprises an amino acid sequence of the formula X¹-X²-X³-X₄-X⁵-X⁶-X⁷-X⁸ (SEQ ID NO:265), wherein X¹=Gly; X²=Phe; X³=Thr; X⁴=Phe; X⁵=Arg; X⁶=Asp or Ser; X⁷=Tyr; and X⁸=Ala or Gly; the HCDR2 comprises an amino acid sequence of the formula X¹-X²-X³-X₄-X⁵-X⁶-X⁷-X⁸ (SEQ ID NO:266), wherein X¹=Ile, X²=Ser, X³=Gly or Tyr, X⁴=Ser or Thr, X⁵=Gly, X⁶=Gly or Ser, X⁷=Asn, and X⁸=Thr or Lys; the HCDR3 comprises an amino acid sequence of the formula X¹-X²-X³-X₄-X⁵-X⁶-X⁷-X⁸-X⁹-X¹⁰-X¹¹-X¹²-X¹³-X¹⁴-X¹⁵-X¹⁶-X¹⁷-X¹⁸ (SEQ ID NO:267) wherein X¹=Ala, X²=Lys, X³=Asp or Glu, X⁴=Gly or Arg, X⁵=Leu or Arg, X⁶=Gly or Ser, X⁷=Ile or Gly, X⁸=Thr or Phe, X⁹=Ile or Asp, X¹⁰=Arg or Tyr, X¹¹=Pro or absent, X¹²=Arg or absent, X¹³=Tyr or absent, X¹⁴=Tyr or absent, X¹⁵=Gly or absent, X¹⁶=Leu or absent, X¹⁷=Asp or absent, and X¹⁸=Val or absent; the LCDR1 comprises an amino acid sequence of the formula X¹-X²-X³-X⁴-X⁵-X⁶-X⁷-X⁸-X⁹-X¹⁰-X¹¹ (SEQ ID NO:268) wherein X¹=Gln, X²=Ser or Val, X³=Ile or Leu, X⁴=Leu or Asn, X⁵=Asn or Tyr, X⁶=Ser or Tyr; X⁷=Ile or absent; X⁸=Gly or absent; X⁹=Tyr or absent; X¹⁰=Asn or absent; and X¹¹=Tyr or absent; the LCDR2 comprises an amino acid sequence of the formula X¹-X²-X³ (SEQ ID NO:269) wherein X¹=Leu or Ala, X²=Ala or Gly, and X³=Ser; and the LCDR3 comprises an amino acid sequence of the formula X¹-X²-X³-X⁴-X⁵-X⁶-X⁷-X⁸-X⁹ (SEQ ID NO:270) wherein X¹=Gln or Met, X²=Gln, X³=Ala or Tyr, X⁴=Leu or Asn, X⁵=Gln or Ser, X⁶=Thr or His, X⁷=Pro, X⁸=Tyr or Trp, and X⁹=Thr.

In another more specific embodiment, the HCDR1 comprises an amino acid sequence of the formula X¹-X²-X³-X⁴-X⁵-X⁶-X⁷-X⁸ (SEQ ID NO:265), wherein X¹=Gly; X²=Phe; X³=Thr; X⁴=Phe; X⁵=Asp or Arg; X⁶=Asp; X⁷=Tyr; and X⁸=Ala; the HCDR2 comprises an amino acid sequence of the formula X¹-X²-X³-X⁴-X⁵-X⁶-X⁷-X⁸ (SEQ ID NO:266), wherein X¹=Ile or Leu, X²=Ser, X³=Gly or Arg, X⁴=Ser or Thr, X⁵=Gly or Ser, X⁶=Gly or Val, X⁷=Ser or Asn, and X⁸=Thr or Ile; the HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸ (SEQ ID NO: 267) wherein X¹=Ala, X²=Lys, X³=Asp or Trp, X⁴=Gly or Arg, X⁵=Leu or Thr, X⁶=Arg or Ser, X⁷=Ile or Gly, X⁸=Thr or Tyr, X⁹=Ile or Phe, X¹⁰=Arg or Asp, X¹¹=Pro, Tyr or absent, X¹²=Arg or absent, X¹³=Tyr or absent, X¹⁴=Tyr or absent, X¹⁵=Gly or absent, X¹⁶=Leu or absent, X¹⁷=Asp or absent, and X¹⁸=Val or absent; the LCDR1 comprises an amino acid sequence of the formula X¹-X²-X³-X₄-X⁵-X⁶-X⁷-X⁸-X⁹-X¹⁰-X¹¹ (SEQ ID NO:268) wherein X¹=Gln, X²=Asp or Ser, X³=Ile or Leu, X⁴=Ser or Leu, X⁵=Tyr or Ile, X⁶=Trp or Ser; X⁷=Ile or absent; X⁸=Gly or absent; X⁹=Tyr or absent; X¹⁰=Asn or absent; and X¹¹=Tyr or absent; the LCDR2 comprises an amino acid sequence of the formula X¹-X²-X³ (SEQ ID NO: 269) wherein X¹=Leu or Val, X²=Ala or Gly, and X³=Ser; and the LCDR3 comprises an amino acid sequence of the formula X¹-X²-X³-X⁴-X⁵-X⁶-X⁷-X⁸-X⁹ (SEQ ID NO:270) wherein X¹=Gln or Met, X²=Gln, X³=Ala, X⁴=Leu or Asn, X⁵=Gln or Ser, X⁶=Thr or Phe, X⁷=Pro, X⁸=Tyr or Ile, and X⁹=Thr.

In a ninth aspect, the invention provides an antibody or antigen-binding fragment comprising HCDR1/HCDR2/HCDR3/LCDR1/LCDR2/LCDR3 sequences from a HCVR and LCVR pair, wherein the HCVR/LCVR sequences are selected from the group consisting of SEQ ID NO: 162/164, 210/212 and 18/20. In a more specific embodiment, heavy and light chain CDR sequences are those contained in HCVR SEQ ID NO: 162 and LCVR SEQ ID NO: 164. In another more specific embodiment, heavy and light chain CDR sequences are those contained in HCVR SEQ ID NO: 18 and LCVR SEQ ID NO:20. In yet another specific embodiment, heavy and light chain CDR sequences are those contained in HCVR SEQ ID NO:210 and LCVR SEQ ID NO:212.

The invention encompasses anti-hIL-4R antibodies having a modified glycosylation pattern. In some applications, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277: 26733). In other applications, modification of a galactosylation can be made in order to modify complement dependent cytotoxicity (CDC).

In a tenth aspect, the invention provides recombinant expression vectors carrying the nucleic acid molecules of the invention, and host cells into which such vectors have been included, as are methods of making the antibodies or antigen-binding fragments of the invention obtained by culturing the host cells of the invention. The host cell may be a prokaryotic or eukaryotic cell, preferably the host cell is an E. coli cell or a mammalian cell, such as a CHO cell.

In an eleventh aspect, the invention features a composition comprising a recombinant human antibody that specifically binds hIL-4R and an acceptable carrier.

In a twelfth aspect, the invention features methods for inhibiting hIL-4 activity using an antibody, or antigen-binding portion thereof, of the invention. In specific embodiments, the antibodies of the invention also block hIL-13/hIL-13R1 complex binding to hIL-4R. In one embodiment, the method comprises contacting hIL-4R with the antibody of the invention, or antigen-binding portion thereof, such that hIL-4 or hIL-4/hIL-13 activity is inhibited. In another embodiment, the method comprises administering an antibody of the invention, or antigen-binding portion thereof, to a human subject suffering from a disorder that is ameliorated by inhibition of hIL-4 or hIL-4/hIL-13 activity. The disorder treated is any disease or condition that is improved, ameliorated, inhibited or prevented by removal, inhibition or reduction of hIL-4 or hIL-4/hIL-13 activity.

IL-4 related disorders which are treated by the antibodies or antibody fragments of the invention include, for example, arthritis (including septic arthritis), herpetiformis, chronic idiopathic urticaria, scleroderma, hypertrophic scarring, Whipple's Disease, benign prostate hyperplasia, lung disorders, such as mild, moderate or severe asthma, inflammatory disorders such as inflammatory bowel disease, allergic reactions, Kawasaki disease, sickle cell disease, Churg-Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

### Definitions

The term “human IL4R” (hIL-4R), as used herein, is intended to refer to a human cytokine receptor that specifically binds interleukin-4 (IL-4), IL-4Rα (SEQ ID NO:274). The term “human interleukin-13” (hIL-13) refers to a cytokine that specifically binds IL-13 receptor, and “hIL-13/hIL-13R1 complex” refers to the complex formed by hIL-13 binding to hIL-13R1 complex, which complex binds hIL-4 receptor to initiate biological activity.

The term “antibody”, as used herein, is intended to refer to immunoglobulin molecules comprising four polypeptide chains, two heavy (H) chains and two light (L) chains inter-connected by disulfide bonds. Each heavy chain comprises a heavy chain variable region (HCVR or VH) and a heavy chain constant region. The heavy chain constant region comprises three domains, CH1, CH2 and CH3. Each light chain comprises a light chain variable region (LCVR or VL) and a light chain constant region. The light chain constant region comprises one domain (CL1). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or simply “antibody portion” or “antibody fragment”), as used herein, refers to one or more fragments of an antibody that retain the ability to specifically bind to an antigen (e.g., hIL-4R). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL1 and CH1 domains; (ii) a F(ab′)₂ fragment, a bivalent fragment comprising two F (ab)′ fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain; and (vi) an CDR. Furthermore, although the two domains of the Fv fragment, VL and VH, are coded for by separate genes, they can be joined, using recombinant methods, by a synthetic linker that enables them to be made as a single contiguous chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883. Such single chain antibodies are also intended to be encompassed within the term “antigen-binding portion” of an antibody. Other forms of single chain antibodies, such as diabodies, are also encompassed (see e.g., Holliger et al. (1993) Proc. Natl. Acad Sci. USA 90:6444-6448).

A “neutralizing” or “blocking” antibody, as used herein, is intended to refer to an antibody whose binding to hIL-4R results in inhibition of the biological activity of hIL-4 and/or hIL-13. This inhibition of the biological activity of hIL-4 and/or IL-13 can be assessed by measuring one or more indicators of hIL-4 and/or hIL-13 biological activity known to the art, such as hlL-4- and/or IL-13-induced cellular activation and hIL-4 binding to hIL-4R (see examples below).

A “CDR” or complementarity determining region is a region of hypervariability interspersed within regions that are more conserved, termed “framework regions” (FR). In different embodiments of the anti-hIL-4R antibody or fragment of the invention, the FRs may be identical to the human germline sequences, or may be naturally or artificially modified.

The term “surface plasmon resonance”, as used herein, refers to an optical phenomenon that allows for the analysis of real-time interactions by detection of alterations in protein concentrations within a biosensor matrix, for example using the BIACORE™ system (Pharmacia Biosensor AB).

The term “epitope” is an antigenic determinant that interacts with a specific antigen binding site in the variable region of an antibody molecule known as a paratope. A single antigen may have more than one epitope. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sufonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each of which is herein incorporated by reference.

### Preparation of Human Antibodies

Methods for generating human antibodies include those described in, for example, U.S. Pat. No. 6,596,541, Green et al. (1994) Nature Genetics 7:13-21), U.S. Pat. Nos. 5,545,807, 6,787,637.

Rodents can be immunized by any method known in the art (see, for example, Harlow and Lane (1988) Antibodies: A Laboratory Manual 1988 Cold Spring Harbor Laboratory; Malik and Lillehoj (1994) Antibody Techniques, Academic Press, CA). Antibodies of the invention are preferably prepared with the use of VELOCIMMUNE™ technology (U.S. Pat. No. 6,596,541). A transgenic mouse in which the endogenous immunoglobulin heavy and light chain variable regions are replaced with the corresponding human variable regions is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable regions of the light and heavy chains may be isolated directly from antigen-specific lymphocytes.

The DNA encoding the variable regions of the heavy and light chains of the antibody are isolated and operably linked to DNA encoding the human heavy and light chain constant regions. The DNA is then expressed in a cell capable of expressing the fully human antibody. In a specific embodiment, the cell is a CHO cell.

Antibodies may be therapeutically useful in blocking a ligand-receptor interaction or inhibiting receptor component interaction, rather than by killing cells through fixation of complement (complement-dependent cytotoxicity) (CDC) and participation antibody-dependent cell-mediated cytotoxicity (ADCC). The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

Human immunoglobulins can exist in two forms that are associated with hinge heterogeneity. In one form, an immunoglobulin molecule comprises a stable four-chain construct of approximately 150-160 kDa in which the dimers are held together by an interchain heavy chain disulfide bond. In a second form, the dimers are not linked via interchain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. In fact, a single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, CH2 or CH3 region that may be desirable, for example, in production, to improve the yield of the desired antibody form.

Initially, high affinity chimeric antibodies are isolated having a human variable region and a mouse constant region. As described below, the antibodies are characterized and selected for desirable characteristics, including binding affinity to hIL-4R, ability to block hIL-4 binding to hIL-4R, and/or selectivity for the human protein. The mouse constant regions are replaced with desired human constant regions to generate the fully human antibodies of the invention, for example wild-type or modified IgG4 or IgG1 (for example, SEQ ID NO:271, 272, 273). While the constant region selected may vary according to specific use, high affinity antigen-binding and target specificity characteristics reside in the variable region.

### Epitope Mapping and Related Technologies

To screen for antibodies that bind to a particular epitope, a routine cross-blocking assay such as that described in Harlow and Lane supra can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science: 9:487-496).

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US Patent Application Publication No. 2004/0101920, herein specifically incorporated by reference in its entirety). Each category may reflect a unique epitope either distinctly different from, or partially overlapping with, an epitope represented by another category. This technology allows rapid filtering of genetically identical antibodies, such that characterization can be focused on genetically distinct antibodies. When applied to hybridoma screening, MAP may facilitate identification of rare hybridoma clones with desired characteristics. MAP may be used to sort the hIL-4R antibodies of the invention into groups of antibodies binding different epitopes.

Agents useful for altering the structure of the immobilized antigen are enzymes, such as, for example, proteolytic enzymes and chemical agents. The antigen protein may be immobilized on either biosensor chip surfaces or polystyrene beads. The latter can be processed with, for example, an assay such as a multiplex LUMINEX™ detection assay (Luminex Corp., TX).

Because of the capacity of LUMINEXT to handle multiplex analysis with up to 100 different types of beads, LUMINEX™ provides almost unlimited antigen surfaces with various modifications, resulting in improved resolution in antibody epitope profiling over a biosensor assay.

### Bispecifics

The antibodies of the present invention may be monospecific, bispecific, or multispecific. Multispecific antibodies may be specific for different epitopes of one target polypeptide or may contain antigen-binding domains specific for more than one target polypeptide. See, e.g., Tutt et al. (1991) J. Immunol. 147:60-69. The human anti-IL-4R antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment, to produce a bispecific or a multispecific antibody with a second binding specificity.

### Therapeutic Administration and Formulations

The invention provides therapeutic compositions comprising the anti-IL-4R antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose may vary depending upon the age and the size of a subject to be administered, target disease, conditions, route of administration, and the like. When the antibody of the present invention is used for treating various conditions and diseases associated with IL-4R, in an adult patient, it is advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used (see Medical Applications of Controlled Release, Langer and Wise (eds.), CRC Pres., Boca Raton, Florida (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984). Other controlled release systems are discussed in the review by Langer (1990) Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to 500 mg per dosage form in a unit dose; especially in the form of injection, it is preferred that the aforesaid antibody is contained in about 5 to 100 mg and in about 10 to 250 mg for the other dosage forms.

Single and combination therapies. The antibodies and antibody fragments of the invention are useful for treating diseases and disorders which are improved, inhibited or ameliorated by reducing IL-4 activity. These disorders include those characterized by abnormal or excess expression of IL-4, or by an abnormal host response to IL-4 production. IL-4 related disorders which are treated by the antibodies or antibody fragments of the include, for example, arthritis (including septic arthritis), herpetiformis, chronic idiopathic urticaria, scleroderma, hypertrophic scarring, Whipple's Disease, benign prostate hyperplasia, pulmonary disorders such as asthma (mild, moderate or severe), inflammatory disorders such as inflammatory bowel disease, allergic reactions, Kawasaki disease, sickle cell disease, Churg-Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, atopic dermatatis, ulcerative colitis, fibrosis, and nephrosis (see U.S. Pat. No. 7,186,809, herein specifically incorporated by reference).

The invention encompasses combination therapies in which the anti-IL-4R antibody or antibody fragment is administered in combination with a second therapeutic agent. Co-administration and combination therapy are not limited to simultaneous administration, but include treatment regimens in which an anti-IL-4R antibody or antibody fragment is administered at least once during a course of treatment that involves administering at least one other therapeutic agent to the patient. A second therapeutic agent may be another IL-4 antagonist, such as another antibody/antibody fragment, or a soluble cytokine receptor, an IgE antagonist, an anti-asthma medication (corticosteroids, non-steroidal agents, beta agonists, leukotriene antagonists, xanthines, fluticasone, salmeterol, albuterol) which may be delivered by inhalation or other appropriate means. In a specific embodiment, the anti-IL-4R antibody or antibody fragment of the invention may be administered with an IL-1 antagonist, such as rilonacept, or an IL-13 antagonist. The second agent may include one or more leukotriene receptor antagonists to treat disorders such as allergic inflammatory diseases, e.g., asthma and allergies. Examples of leukotriene receptor antagonists include but are not limited to montelukast, pranlukast, and zafirlukast. The second agent may include a cytokine inhibitor such as one or more of a TNF (etanercept, ENBREL™), IL-9, IL-5 or IL-17 antagonist.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Human IL-4 Receptor

VELOCIMMUNE™ mice (Regeneron Pharmaceuticals, Inc.; U.S. Pat. No. 6,596,541) were immunized with human IL-4R (hIL-4R, SEQ ID NO:274) or a combination of hIL-4R and monkey (Macaca fascicularis) IL-4R (mfIL-4R, SEQ ID NO:275) protein or DNA. To obtain optimal immune response, animals were subsequently boosted every 3-4 weeks and bleeds obtained 10 days after each boost for assessment of progression of anti-antigen response.

When the mice attained maximum immune response, antibody-expressing B cells were harvested and fused with mouse myeloma cells to form hybridomas. Alternatively, antigen-specific antibodies may be isolated directly from the B cells without fusion to myeloma cells, as described in U.S. Patent Publication 2007/0280945A1, herein specifically incorporated by reference in its entirety. Stable recombinant antibody-expressing CHO cell lines were established from the isolated proper recombinants. Functionally desirable monoclonal antibodies were selected by screening conditioned media of the hybridomas or transfected cells for specificity, antigen-binding affinity, and potency in blocking hIL-4 binding to hIL-4R (described below).

### Example 2. Antigen Binding Affinity Determination

Binding affinity (K_{D}) of selected antibodies with respect to hIL-4R at either 25° C. or 37° C. was determined using a real-time biosensor surface plasmon resonance assay (BIACORE™ 2000). Briefly, antibody was captured on a goat anti-hFc polyclonal antibody surface created through direct coupling to a BIACORE™ chip to form a captured antibody surface. Various concentrations (ranging from 50 nM to 12.5 nM) of monomeric hIL-4R (R&D Systems) or dimeric hIL-4R-mFc were injected over the captured antibody surface at 10 μl/min for 2.5 min at either 25° C. or 37° C. Binding of antigen to antibody and dissociation of the bound complex, were monitored in real time. Equilibrium dissociation constants (K_{D}) and dissociation rate constants were ascertained by performing kinetic analysis using BIA evaluation software. BIA evaluation software was also used to calculate the half-life of antigen/antibody complex dissociation (T_{1/2}). Results are shown in Table 1. NB: No antibody-antigen binding was observed under the experimental condition. Control: a fully human anti-IL-4R antibody (U.S. Pat. No. 7,186,809; SEQ ID NOs: 10 and 12).

TABLE 1

| | 25° C. | | | | 37° C. | | | |
|---|---|---|---|---|---|---|---|---|
| | Monomeric | | Dimeric | | Monomeric | | Dimeric | |
| Antibody | K_{D} (pM) | T_{1/2} (min) | K_{D} (pM) | T_{1/2} (min) | K_{D} (pM) | T_{1/2} (min) | K_{D} (pM) | T_{1/2} (min) |
| Control | 1100 | 18 | 94 | 186 | 3970 | 4 | 114 | 158 |
| H4H083P | 48 | 361 | 28 | 245 | 183 | 87 | 38.1 | 163 |
| H4H094P | NB | — | NB | — | NB | — | NB | — |
| H4H095P | 274 | 131 | 302 | 156 | 437 | 49 | 314 | 116 |
| H4H098P | 94.1 | 243 | 67.6 | 237 | 157 | 129 | 38.8 | 158 |
| H4H099P | NB | — | NB | — | NB | — | NB | — |

Binding affinity (K_{D}) of selected antibodies with respect to monkey (Macaca fascicularis) IL-4R (mfIL-4R) at either 25° C. or 37° C. was also determined using a real-time biosensor surface plasmon resonance assay described above with various concentrations (ranging from 100 nM to 25 nM) of monomeric mfIL-4R-myc-myc-his (mfIL-4R-mmh) or dimeric mfIL-4R-mFc. Only antibody H4H098P was able to bind both monomeric and dimeric mfIL-4R at 25° C. with K_{D} of 552 nM and 9.08 nM, respectively. In addition, antibody H4H098P also binds to dimeric mfIL-4R at 37° C. with a K_{D} of 24.3 nM. H4H083P had very weak binding to dimeric mfIL-4R.

Antibody-antigen binding affinity was also assessed using an ELISA-based solution competition assay. Briefly, a 96-well MAXISORPT plate was first coated with 5 μg/ml avidin overnight followed by BSA blocking for 1 hr. The avidin-coated plate was then incubated with 250 ng/ml biotin-hIL4 for 2 hr. The plate was used to measure either free hIL-4R-mFc (dimeric hIL-4R) or free hIL-4R-myc-myc-his (hIL4R-mmh, monomeric hIL4R) in the antibody titration sample solutions. To make the antibody titration sample, a constant amount either 25 PM of hIL-4R-mFc or 200 pM of hIL-4R-mmh was premixed with varied amounts of antibody, ranging from 0 to about 10 nM in serial dilutions, followed by 1 hr incubation at room temperature to allow antibody-antigen-binding to reach equilibrium. The equilibrated sample solutions were then transferred to the hIL-4 coated plates for measurement of either free hIL-4R-mFc or free hIL-4R-mmh. After 1 hr binding, the plate was washed and bound hIL-4R-mFc was detected using either an HRP-conjugated mouse anti-mFc polyclonal antibody or an HRP-conjugated goat anti-myc polyclonal antibodies. IC₅₀ values were determined (Table 2).

TABLE 2

IC₅₀ (pM)

| Antibody | 25 pM hIL-4R-mFc | 200 pM hIL-4R-mmh |
|---|---|---|
| Control | 8.2 | 87 |
| H4H083P | 9.6 | 80 |
| H4H094P | >10,000 | >10,000 |
| H4H095P | 40 | 90 |
| H4H098P | 8.8 | 74 |
| H4H099P | >10,000 | >10,000 |

The ELISA-based solution competition assay was also used to determine the cross reactivity of the antibodies to monkey IL-4R. Antibody H4H098P exhibits an IC₅₀ for mfIL-4R-mFc of 300 PM and an IC₅₀ for mfIL-4R-mmh of 20 nM.

### Example 3. Neutralization of Biological Effect of hIL-4 and hIL-13 In Vitro

A bioassay was developed to determine the purified anti-hIL-4R antibodies to neutralize hIL-4R-mediated cellular function in vitro using an engineered HK293 cell line that contains human STAT6 and a STAT6 luciferase reporter. Inhibition of hIL-4R-inducible luciferase activity was determined as follows: Cells were seeded onto 96-well plates at 1×10⁴ cells/well in media and incubated overnight at 37° C., 5% CO₂. Antibody proteins ranging from 0 to 20 nM in serial dilutions were added to the cells along with either 10 PM hIL-4 or 40 PM of hIL-13. Cells were then incubated at 37° C., 5% CO₂ for 6 hrs. The extent of cellular response was measured in a luciferase assay (Promega Biotech). Results are shown in Table 3. NB: Luciferase activity was not blocked under the experimental condition described above. In addition, H4H098P was able to block mfIL-4R-mediated cellular function in the presence of 360 fM mfIL-4 with an IC₅₀ of 150 nM.

TABLE 3

IC₅₀ (pM)

| Antibody | 10 pM hIL-4 | 40 pM hIL-13 |
|---|---|---|
| Control | 47 | 38 |
| H4H083P | 25 | 19 |
| H4H094P | NB | NB |
| H4H095P | 98 | 86 |
| H4H098P | 27 | 25 |
| H4H099P | NB | 11,000 |

1. An antibody or antigen-binding fragment thereof, that specifically binds human interleukin-4 receptor (hIL-4R) (SEQ ID NO:274), comprising a heavy chain variable region (HCVR) and a light chain variable region (LCVR), wherein the antibody or antigen-binding fragment comprises:
(a) an HCVR having an amino acid sequence shown in SEQ ID NO:162 and a LCVR having an amino acid sequence shown in SEQ ID NO:164, characterized by an affinity for hIL-4R (K_{D}) of about 100 pM or less;
(b) an HCVR having an amino acid sequence shown in SEQ ID NO:18 and a LCVR having an amino acid sequence shown in SEQ ID NO:20, characterized by an affinity for hIL-4R (K_{D}) of about 300 pM or less; or
(c) an HCVR having an amino acid sequence shown in SEQ ID NO:210 and a LCVR having an amino acid sequence shown in SEQ ID NO:212, characterized by an affinity for hIL-4R (K_{D}) of about 50 pM or less.

2. (canceled)

3. An antibody or antigen-binding fragment of an antibody which specifically binds human IL-4R,
comprising heavy chain complementarity determining region 1 (HCDR1), 2 (HCDR2), 3 (HCDR3) and light chain complementarity determining region 1 (LCDR1), 2 (LCDR2), 3 (LCDR3), wherein
HCDR1 comprises an amino acid sequence of the formula X¹-X²-X³-X⁴-X⁵-X⁶-X⁷-X⁸ (SEQ ID NO:265), wherein X¹=Gly; X²=Phe; X³=Thr; X⁴=Phe; X⁵=Asp or Arg; X⁶=Asp or Ser; X⁷=Tyr; and X⁸=Ala or Gly;
HCDR2 comprises an amino acid sequence of the formula X¹-X²-X³-X⁴-X⁵-X⁶-X⁷-X⁸ (SEQ ID NO:266), wherein X¹=Ile or Leu, X²=Ser, X³=Gly, Tyr or Arg, X⁴=Ser, Asp or Thr, X⁵=Gly or Ser, X⁶=Gly, Ser or Val, X⁷=Ser or Asn, and X⁸=Thr, Lys or Ile;
HCDR3 comprises an amino acid sequence of the formula X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸ (SEQ ID NO:267) wherein X¹=Ala, X²=Lys, X³=Asp, Glu or Trp, X⁴=Gly or Arg, X⁵=Leu, Thr or Arg, X⁶=Gly, Arg or Ser, X⁷=Ile or Gly, X⁸=Thr, Phe or Tyr, X⁹=Ile, Asp or Phe, X¹⁰=Arg, Tyr or Asp, X¹¹-Pro, Tyr or absent, X¹²=Arg or absent, X¹³=Tyr or absent, X¹⁴=Tyr or absent, X¹⁵=Gly or absent, X¹⁶=Leu or absent, X¹⁷=Asp or absent, and X¹⁸=Val or absent;
LCDR1 comprises an amino acid sequence of the formula X¹-X²-X³-X⁴-X⁵-X⁶-X⁷-X⁸-X⁹-X¹⁰-X¹¹ (SEQ ID NO:268) wherein X¹=Gln, X²=Asp, Ser or Val, X³=Ile or Leu, X⁴=Ser, Leu or Asn, X⁵=Asn, Tyr or Ile, X⁶=Trp, Ser or Tyr; X⁷=Ile or absent; X⁸=Gly or absent; X⁹=Tyr or absent; X¹⁰=Asn or absent; and X¹¹=Tyr or absent;
LCDR2 comprises an amino acid sequence of the formula X¹-X²-×3 (SEQ ID NO: 269) wherein X¹=Leu, Ala or Val, X²=Ala or Gly, and X³=Ser; and
LCDR3 comprises an amino acid sequence of the formula X¹-X²-X³-X⁴-X⁵-X⁶-X⁷-X⁸-X⁹ (SEQ ID NO:270) wherein X¹=Gln or Met, X²=Gln, X³=Ala or Tyr, X⁴=Leu or Asn, X⁵=Gln or Ser, X⁶=Thr, Phe or His, X⁷=Pro, X⁸=Tyr, Ile or Trp, and X⁹=Thr.

4. (canceled)

5. A nucleic acid sequence encoding the HCDR1, HCDR2, HCDR3, LCDR1, LCDR2 and LCDR3 sequences of the antibodies or antigen-binding fragments of claim 1.

6. A vector comprising the nucleic acid sequence of claim 5.

7. A host-vector system for the production of an antibody or antigen-binding fragment of an antibody, comprising the vector of claim 6.

8. A method of producing an antibody or antigen-binding fragment thereof which specifically binds human interleukin-4 receptor alpha (hIL-4R), comprising growing cells of the host-vector system of claim 7 under conditions in which the antibody or fragment is expressed, and recovering the anti-hIL-4 antibody expressed.

9-13. (canceled)

14. A method of treating a disease or disorder, wherein the disease or disorder is improved, ameliorated or inhibited by removal, inhibition or reduction of human interleukin-4 (hIL-4) activity, said method comprising administering the antibody or antigen-binding fragment of claim 1 to a patient in need thereof.

15-16. (canceled)

17. A method of treating a disease or disorder that is improved, ameliorated or inhibited by removal, inhibition or reduction of human interleukin-4 (hIL-4) activity, the method comprising:
administering to a patient in need thereof an antibody or antigen-binding fragment thereof that specifically binds to human interleukin-4 receptor (hIL-4R), wherein the antibody or antigen-binding fragment thereof comprises:
(i) a heavy chain complementarity determining region (HCDR) 1 sequence comprising SEQ ID NO: 148, an HCDR2 sequence comprising SEQ ID NO: 150, an HCDR3 sequence comprising SEQ ID NO:152, a light chain complementarity determining region (LCDR) 1 sequence comprising SEQ ID NO:156, an LCDR2 sequence comprising SEQ ID NO:158, and an LCDR3 sequence comprising SEQ ID NO: 160; or
(ii) an HCDR1 sequence comprising SEQ ID NO:196, an HCDR2 sequence comprising SEQ ID NO: 198, an HCDR3 sequence comprising SEQ ID NO:200, an LCDR1 sequence comprising SEQ ID NO:204, an LCDR2 sequence comprising SEQ ID NO: 206, and an LCDR3 sequence comprising SEQ ID NO:208; or
(iii) an HCDR1 sequence comprising SEQ ID NO:4, an HCDR2 sequence comprising SEQ ID NO:6, an HCDR3 sequence comprising SEQ ID NO:8, an LCDR1 sequence comprising SEQ ID NO: 12, an LCDR2 sequence comprising SEQ ID NO:14, and an LCDR3 sequence comprising SEQ ID NO: 16;
wherein the disease or disorder is ulcerative colitis or inflammatory bowel disease.

18. The method of claim 17, wherein the disease or disorder is ulcerative colitis.

19. The method of claim 17, wherein the disease or disorder is inflammatory bowel disease.

20. The method of claim 17, wherein the antibody or antigen-binding fragment thereof comprises:
(i) a heavy chain variable region (HCVR) comprising the amino acid sequence of SEQ ID NO: 162; or
(ii) an HCVR comprising the amino acid sequence of SEQ ID NO:210; or
(iii) an HCVR comprising the amino acid sequence of SEQ ID NO:18.

21. The method of claim 17, wherein the antibody or antigen-binding fragment thereof comprises:
(i) a light chain variable region (LCVR) comprising the amino acid sequence of SEQ ID NO: 164; or
(ii) an LCVR comprising the amino acid sequence of SEQ ID NO:212; or
(iii) an LCVR comprising the amino acid sequence of SEQ ID NO:20.

22. The method of claim 17, wherein the antibody or antigen-binding fragment thereof comprises:
(i) an HCVR comprising the amino acid sequence of SEQ ID NO: 162 and an LCVR comprising the amino acid sequence of SEQ ID NO:164; or
(ii) an HCVR comprising the amino acid sequence of SEQ ID NO:210 and an LCVR comprising the amino acid sequence of SEQ ID NO:212; or
(iii) an HCVR comprising the amino acid sequence of SEQ ID NO:18 and an LCVR comprising the amino acid sequence of SEQ ID NO:20.

23. The method of claim 17, wherein the antibody or antigen-binding fragment thereof is a full length IgG4 antibody.

24. The method of claim 17, wherein the antibody or antigen-binding fragment thereof is administered subcutaneously.


SEQUENCE LISTING (275 sequences)

SEQ ID NO: 1 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttccgctcttatggcatgcactgggtccgccaggctccaggcaaggggctggagtgggtggcggtcatatcatatgatggaagtaataaatattatatagactccgtgaagggccgattcaccatctccagagacaattccaagaacacgctgaatctgcaaatgaacagcctgagacttgaggacacggctgtatattactgtgcgaaagaggggagggggggatttgactactggggccagggaatcccggtcaccgtctcctca

SEQ ID NO: 2 - protein, 117 aa, synthetic construct
QVQLVESGGGVVQPGRSLRLSCAASGFTFRSYGMHWVRQAPGKGLEWVAVISYDGSNKYYIDSVKGRFTISRDNSKNTLNLQMNSLRLEDTAVYYCAKEGRGGFDYWGQGIPVTVSS

SEQ ID NO: 3 - DNA, 24 bp, synthetic construct
  source 1..24: mol_type = other DNA
ggattcaccttccgctcttatggc

SEQ ID NO: 4 - protein, 8 aa, synthetic construct
GFTFRSYG

SEQ ID NO: 5 - DNA, 24 bp, synthetic construct
  source 1..24: mol_type = other DNA
atatcatatgatggaagtaataaa

SEQ ID NO: 6 - protein, 8 aa, synthetic construct
ISYDGSNK

SEQ ID NO: 7 - DNA, 30 bp, synthetic construct
  source 1..30: mol_type = other DNA
gcgaaagaggggagggggggatttgactac

SEQ ID NO: 8 - protein, 10 aa, synthetic construct
AKEGRGGFDY

SEQ ID NO: 9 - DNA, 324 bp, synthetic construct
  source 1..324: mol_type = other DNA
gacatccagatgacccagtctccatcctcactgtctgcatctgtaggagacagagtcaccatcacttgtcgggcgagtcaggtcataaacaattatttagcctggtttcagcagaaaccagggaaagtccctaagtccctgatccatgctgcatccagtttacaaagtggggtcccatcaaagttcagcggcagtggatctgggacagatttcactctcaccatcagcagcctgcagcctgaagattttgcaacttattactgccaacagtataatagtcacccgtggacgttcggccaagggaccaaggtggaaatcaaacga

SEQ ID NO: 10 - protein, 108 aa, synthetic construct
DIQMTQSPSSLSASVGDRVTITCRASQVINNYLAWFQQKPGKVPKSLIHAASSLQSGVPSKFSGSGSGTDFTLTISSLQPEDFATYYCQQYNSHPWTFGQGTKVEIKR

SEQ ID NO: 11 - DNA, 18 bp, synthetic construct
  source 1..18: mol_type = other DNA
caggtcataaacaattat

SEQ ID NO: 12 - protein, 6 aa, synthetic construct
QVINNY

SEQ ID NO: 13-14 - skipped (2 entries)

SEQ ID NO: 15 - DNA, 27 bp, synthetic construct
  source 1..27: mol_type = other DNA
caacagtataatagtcacccgtggacg

SEQ ID NO: 16 - protein, 9 aa, synthetic construct
QQYNSHPWT

SEQ ID NO: 17 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttccgctcttatggcatgcactgggtccgccaggctccaggcaaggggctggagtgggtggcggtcatatcatatgatggaagtaataaatattatatagactccgtgaagggccgattcaccatctccagagacaattccaagaacacgctgaatctgcaaatgaacagcctgagacttgaggacacggctgtatattactgtgcgaaagaggggagggggggatttgactactggggccagggaaccctggtcaccgtctcctca

SEQ ID NO: 18 - protein, 117 aa, synthetic construct
QVQLVESGGGVVQPGRSLRLSCAASGFTFRSYGMHWVRQAPGKGLEWVAVISYDGSNKYYIDSVKGRFTISRDNSKNTLNLQMNSLRLEDTAVYYCAKEGRGGFDYWGQGTLVTVSS

SEQ ID NO: 19 - DNA, 321 bp, synthetic construct
  source 1..321: mol_type = other DNA
gacatccagatgacccagtctccatcctcactgtctgcatctgtaggagacagagtcaccatcacttgtcgggcgagtcaggtcataaacaattatttagcctggtttcagcagaaaccagggaaagtccctaagtccctgatccatgctgcatccagtttacaaagtggggtcccatcaaagttcagcggcagtggatctgggacagatttcactctcaccatcagcagcctgcagcctgaagattttgcaacttattactgccaacagtataatagtcacccgtggacgttcggccaagggaccaaggtggaaatcaaa

SEQ ID NO: 20 - protein, 107 aa, synthetic construct
DIQMTQSPSSLSASVGDRVTITCRASQVINNYLAWFQQKPGKVPKSLIHAASSLQSGVPSKFSGSGSGTDFTLTISSLQPEDFATYYCQQYNSHPWTFGQGTKVEIK

SEQ ID NO: 21 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttccgctcttatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagaggggagggggggatttgactactggggccagggaaccctggtcaccgtctcctca

SEQ ID NO: 22 - protein, 117 aa, synthetic construct
QVQLVESGGGVVQPGRSLRLSCAASGFTFRSYGMHWVRQAPGKGLEWVAVISYDGSNKYYADSVKGRFTISRDNSKNTLYLQMNSLRAEDTAVYYCAKEGRGGFDYWGQGTLVTVSS

SEQ ID NO: 23 - DNA, 322 bp, synthetic construct
  source 1..322: mol_type = other DNA
gacatccagatgacccagtctccatcctcactgtctgcatctgtaggagacagagtcaccatcacttgtcgggcgagtcaggtcataaacaattatttagcctggtttcagcagaaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagtcacccgtggacgttcggccaagggaccaaggtggaaatcaaac

SEQ ID NO: 24 - protein, 107 aa, synthetic construct
DIQMTQSPSSLSASVGDRVTITCRASQVINNYLAWFQQKPGKAPKSLIYAASSLQSGVPSRFSGSGSGTDFTLTISSLQPEDFATYYCQQYNSHPWTFGQGTKVEIK

SEQ ID NO: 25 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttcagaagctatggcatacactgggtccgccaggctccaggcaaggggctggagtgggtggcagttatatcatatgatggaagtaataaatactatgcagactccgtgaagggccgattcaccatctccagagacaattccaagaacacactgtatctgcaaatgaacagcctgataactgaggacacggctgtgtattattgtgtgaaagaggggaggggggggtttgactactggggccagggaaccacggtcaccgtctcctca

SEQ ID NO: 26 - protein, 117 aa, synthetic construct
QVQLVESGGGVVQPGRSLRLSCAASGFTFRSYGIHWVRQAPGKGLEWVAVISYDGSNKYYADSVKGRFTISRDNSKNTLYLQMNSLITEDTAVYYCVKEGRGGFDYWGQGTTVTVSS

SEQ ID NO: 27 - DNA, 24 bp, synthetic construct
  source 1..24: mol_type = other DNA
ggattcaccttcagaagctatggc

SEQ ID NO: 28 - protein, 8 aa, synthetic construct
GFTFRSYG

SEQ ID NO: 29 - DNA, 24 bp, synthetic construct
  source 1..24: mol_type = other DNA
atatcatatgatggaagtaataaa

SEQ ID NO: 30 - protein, 8 aa, synthetic construct
ISYDGSNK

SEQ ID NO: 31 - DNA, 30 bp, synthetic construct
  source 1..30: mol_type = other DNA
gtgaaagaggggaggggggggtttgactac

SEQ ID NO: 32 - protein, 10 aa, synthetic construct
VKEGRGGFDY

SEQ ID NO: 33 - DNA, 324 bp, synthetic construct
  source 1..324: mol_type = other DNA
gacatccagatgacccagtctccatcctcactgtctgcatctgtaggagacagagtcaccatcacttgtcgggcgagtcaggtcattaataattatttagcctggtttcagcagaaaccagggaaagtccctaagtccctgatccatgctgcatccagtttgcaaagaggggtcccatcaaagttcagcggcagtggatctgggacagatttcactctcaccatcaacagcctgcagcctgaagattttgcaacttattactgccaacaatataatagttacccgtggacgttcggccaagggaccaaggtggaaatcaaacga

SEQ ID NO: 34 - protein, 108 aa, synthetic construct
DIQMTQSPSSLSASVGDRVTITCRASQVINNYLAWFQQKPGKVPKSLIHAASSLQRGVPSKFSGSGSGTDFTLTINSLQPEDFATYYCQQYNSYPWTFGQGTKVEIKR

SEQ ID NO: 35 - DNA, 18 bp, synthetic construct
  source 1..18: mol_type = other DNA
caggtcattaataattat

SEQ ID NO: 36 - protein, 6 aa, synthetic construct
QVINNY

SEQ ID NO: 37-38 - skipped (2 entries)

SEQ ID NO: 39 - DNA, 27 bp, synthetic construct
  source 1..27: mol_type = other DNA
caacaatataatagttacccgtggacg

SEQ ID NO: 40 - protein, 9 aa, synthetic construct
QQYNSYPWT

SEQ ID NO: 41 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttcagaagctatggcatacactgggtccgccaggctccaggcaaggggctggagtgggtggcagttatatcatatgatggaagtaataaatactatgcagactccgtgaagggccgattcaccatctccagagacaattccaagaacacactgtatctgcaaatgaacagcctgataactgaggacacggctgtgtattattgtgtgaaagaggggaggggggggtttgactactggggccagggaaccctggtcaccgtctcctca

SEQ ID NO: 42 - protein, 117 aa, synthetic construct
QVQLVESGGGVVQPGRSLRLSCAASGFTFRSYGIHWVRQAPGKGLEWVAVISYDGSNKYYADSVKGRFTISRDNSKNTLYLQMNSLITEDTAVYYCVKEGRGGFDYWGQGTLVTVSS

SEQ ID NO: 43 - DNA, 321 bp, synthetic construct
  source 1..321: mol_type = other DNA
gacatccagatgacccagtctccatcctcactgtctgcatctgtaggagacagagtcaccatcacttgtcgggcgagtcaggtcattaataattatttagcctggtttcagcagaaaccagggaaagtccctaagtccctgatccatgctgcatccagtttgcaaagaggggtcccatcaaagttcagcggcagtggatctgggacagatttcactctcaccatcaacagcctgcagcctgaagattttgcaacttattactgccaacaatataatagttacccgtggacgttcggccaagggaccaaggtggaaatcaaa

SEQ ID NO: 44 - protein, 107 aa, synthetic construct
DIQMTQSPSSLSASVGDRVTITCRASQVINNYLAWFQQKPGKVPKSLIHAASSLQRGVPSKFSGSGSGTDFTLTINSLQPEDFATYYCQQYNSYPWTFGQGTKVEIK

SEQ ID NO: 45 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttcagaagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgtgaaagaggggaggggggggtttgactactggggccagggaaccctggtcaccgtctcctca

SEQ ID NO: 46 - protein, 117 aa, synthetic construct
QVQLVESGGGVVQPGRSLRLSCAASGFTFRSYGMHWVRQAPGKGLEWVAVISYDGSNKYYADSVKGRFTISRDNSKNTLYLQMNSLRAEDTAVYYCVKEGRGGFDYWGQGTLVTVSS

SEQ ID NO: 47 - DNA, 322 bp, synthetic construct
  source 1..322: mol_type = other DNA
gacatccagatgacccagtctccatcctcactgtctgcatctgtaggagacagagtcaccatcacttgtcgggcgagtcaggtcattaataattatttagcctggtttcagcagaaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacaatataatagttacccgtggacgttcggccaagggaccaaggtggaaatcaaac

SEQ ID NO: 48 - protein, 107 aa, synthetic construct
DIQMTQSPSSLSASVGDRVTITCRASQVINNYLAWFQQKPGKAPKSLIYAASSLQSGVPSRFSGSGSGTDFTLTISSLQPEDFATYYCQQYNSYPWTFGQGTKVEIK

SEQ ID NO: 49 - DNA, 375 bp, synthetic construct
  source 1..375: mol_type = other DNA
caggtgcagctggtggagtctgggggaggcttggaacagccgggggggtccttgagactctcctgtgcaggctctggattcacgtttagagactatgccatgacctgggtccgccaggctccagggaaggggctggagtgggtcgcatcgattagtggttccggtggtaacacatacttcgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggtccacggtcaccgtctcctca

SEQ ID NO: 50 - protein, 125 aa, synthetic construct
QVQLVESGGGLEQPGGSLRLSCAGSGFTFRDYAMTWVRQAPGKGLEWVASISGSGGNTYFADSVKGRFTISRDNSKNTLYLQMNSLRAEDTAVYYCAKDRLSITIRPRYYGLDVWGQGSTVTVSS

SEQ ID NO: 51 - DNA, 24 bp, synthetic construct
  source 1..24: mol_type = other DNA
ggattcacgtttagagactatgcc

SEQ ID NO: 52 - protein, 8 aa, synthetic construct
GFTFRDYA

SEQ ID NO: 53 - DNA, 24 bp, synthetic construct
  source 1..24: mol_type = other DNA
attagtggttccggtggtaacaca

SEQ ID NO: 54 - protein, 8 aa, synthetic construct
ISGSGGNT

SEQ ID NO: 55 - DNA, 54 bp, synthetic construct
  source 1..54: mol_type = other DNA
gcgaaagatcgactctctataacaattcgcccacgctattatggtttggacgtc

SEQ ID NO: 56 - protein, 18 aa, synthetic construct
AKDRLSITIRPRYYGLDV

SEQ ID NO: 57 - DNA, 324 bp, synthetic construct
  source 1..324: mol_type = other DNA
gacatccagatgacccagtctccatcctcactgtctgcatctgttggagacagagtcaccatcacttgtcgggcgagtcaggccattaacaatcatttagcctggtttcagcagaaaccagggaaagcccctaagtccctgatctttgctgtatccagtttgcaaagtggggtcccatcaaagttcagcggcagtggatctgggacagacttcactctcaccatcagcagcctgcagcctgaagattttgcaacttattactgccaacagtataatagttacccgtggacgttcggccaagggaccaaggtggaaatcaaacga

SEQ ID NO: 58 - protein, 108 aa, synthetic construct
DIQMTQSPSSLSASVGDRVTITCRASQAINNHLAWFQQKPGKAPKSLIFAVSSLQSGVPSKFSGSGSGTDFTLTISSLQPEDFATYYCQQYNSYPWTFGQGTKVEIKR

SEQ ID NO: 59 - DNA, 18 bp, synthetic construct
  source 1..18: mol_type = other DNA
caggccattaacaatcat

SEQ ID NO: 60 - protein, 6 aa, synthetic construct
QAINNH

SEQ ID NO: 61-62 - skipped (2 entries)

SEQ ID NO: 63 - DNA, 27 bp, synthetic construct
  source 1..27: mol_type = other DNA
caacagtataatagttacccgtggacg

SEQ ID NO: 64 - protein, 9 aa, synthetic construct
QQYNSYPWT

SEQ ID NO: 65 - DNA, 372 bp, synthetic construct
  source 1..372: mol_type = other DNA
gaggtgcagctggtggagtctgggggaggcttggaacagccgggggggtccttgagactctcctgtgcaggctctggattcacgtttagagactatgccatgacctgggtccgccaggctccagggaaggggctggagtgggtcgcatcgattagtggttccggtggtaacacatacttcgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggaccacggtcaccgtctcc

SEQ ID NO: 66 - protein, 124 aa, synthetic construct
EVQLVESGGGLEQPGGSLRLSCAGSGFTFRDYAMTWVRQAPGKGLEWVASISGSGGNTYFADSVKGRFTISRDNSKNTLYLQMNSLRAEDTAVYYCAKDRLSITIRPRYYGLDVWGQGTTVTVS

SEQ ID NO: 67 - DNA, 321 bp, synthetic construct
  source 1..321: mol_type = other DNA
gacatccagatgacccagtctccatcctcactgtctgcatctgttggagacagagtcaccatcacttgtcgggcgagtcaggccattaacaatcatttagcctggtttcagcagaaaccagggaaagcccctaagtccctgatctttgctgtatccagtttgcaaagtggggtcccatcaaagttcagcggcagtggatctgggacagacttcactctcaccatcagcagcctgcagcctgaagattttgcaacttattactgccaacagtataatagttacccgtggacgttcggccaagggaccaaggtggaaatcaaa

SEQ ID NO: 68 - protein, 107 aa, synthetic construct
DIQMTQSPSSLSASVGDRVTITCRASQAINNHLAWFQQKPGKAPKSLIFAVSSLQSGVPSKFSGSGSGTDFTLTISSLQPEDFATYYCQQYNSYPWTFGQGTKVEIK

SEQ ID NO: 69 - DNA, 373 bp, synthetic construct
  source 1..373: mol_type = other DNA
gaggtgcagctggtggagtctgggggaggcttggtacagcctggggggtccctgagactctcctgtgcagcctctggattcacgtttagagactatgccatgagctgggtccgccaggctccagggaaggggctggagtgggtctcagctattagtggttccggtggtaacacatactacgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggaccacggtcaccgtctcct

SEQ ID NO: 70 - protein, 124 aa, synthetic construct
EVQLVESGGGLVQPGGSLRLSCAASGFTFRDYAMSWVRQAPGKGLEWVSAISGSGGNTYYADSVKGRFTISRDNSKNTLYLQMNSLRAEDTAVYYCAKDRLSITIRPRYYGLDVWGQGTTVTVS

SEQ ID NO: 71 - DNA, 322 bp, synthetic construct
  source 1..322: mol_type = other DNA
gacatccagatgacccagtctccatcctcactgtctgcatctgtaggagacagagtcaccatcacttgtcgggcgagtcaggccattaacaatcatttagcctggtttcagcagaaaccagggaaagcccctaagtccctgatctatgctgtatccagtttgcaaagtggggtcccatcaaggttcagcggcagtggatctgggacagatttcactctcaccatcagcagcctgcagcctgaagattttgcaacttattactgccaacagtataatagttacccgtggacgttcggccaagggaccaaggtggaaatcaaac

SEQ ID NO: 72 - protein, 107 aa, synthetic construct
DIQMTQSPSSLSASVGDRVTITCRASQAINNHLAWFQQKPGKAPKSLIYAVSSLQSGVPSRFSGSGSGTDFTLTISSLQPEDFATYYCQQYNSYPWTFGQGTKVEIK

SEQ ID NO: 73 - DNA, 375 bp, synthetic construct; identical to SEQ ID NO: 49
  source 1..375: mol_type = other DNA

SEQ ID NO: 74 - protein, 125 aa, synthetic construct; identical to SEQ ID NO: 50

SEQ ID NO: 75 - DNA, 24 bp, synthetic construct
  source 1..24: mol_type = other DNA
ggattcacgtttagagactatgcc

SEQ ID NO: 76 - protein, 8 aa, synthetic construct
GFTFRDYA

SEQ ID NO: 77 - DNA, 24 bp, synthetic construct
  source 1..24: mol_type = other DNA
attagtggttccggtggtaacaca

SEQ ID NO: 78 - protein, 8 aa, synthetic construct
ISGSGGNT

SEQ ID NO: 79 - DNA, 54 bp, synthetic construct; identical to SEQ ID NO: 55
  source 1..54: mol_type = other DNA

SEQ ID NO: 80 - protein, 18 aa, synthetic construct
AKDRLSITIRPRYYGLDV

SEQ ID NO: 81 - DNA, 339 bp, synthetic construct
  source 1..339: mol_type = other DNA
gaaatagtgttgacgcagtctccactctccctgcccgtcacccctggagagccggcctccatctcctgcaggtctagtcagagcctcctgtatagtattggatacaactatttggattggtacctgcagaagtcagggcagtctccacagctccttatctatttgggttctaatcgggcctccggggtccctgacaggttcagtggcagtggatcaggcacagattttacactgaaaatcagcagagtggaggctgaggatgttgggttttattactgcatgcaagctctacaaactccgtacacttttggcccggggaccaagctggagatcaaacga

SEQ ID NO: 82 - protein, 113 aa, synthetic construct
EIVLTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGPGTKLEIKR

SEQ ID NO: 83 - DNA, 33 bp, synthetic construct
  source 1..33: mol_type = other DNA
cagagcctcctgtatagtattggatacaactat

SEQ ID NO: 84 - protein, 11 aa, synthetic construct
QSLLYSIGYNY

SEQ ID NO: 85-86 - skipped (2 entries)

SEQ ID NO: 87 - DNA, 27 bp, synthetic construct
  source 1..27: mol_type = other DNA
atgcaagctctacaaactccgtacact

SEQ ID NO: 88 - protein, 9 aa, synthetic construct
MQALQTPYT

SEQ ID NO: 89 - DNA, 372 bp, synthetic construct; identical to SEQ ID NO: 65
  source 1..372: mol_type = other DNA

SEQ ID NO: 90 - protein, 124 aa, synthetic construct; identical to SEQ ID NO: 66

SEQ ID NO: 91 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgtatagtattggatacaactatttggattggtacctgcagaagtcagggcagtctccacagctccttatctatttgggttctaatcgggcctccggggtccctgacaggttcagtggcagtggatcaggcacagattttacactgaaaatcagcagagtggaggctgaggatgttgggttttattactgcatgcaagctctacaaactccgtacacttttggcccggggaccaagctggagatcaaa

SEQ ID NO: 92 - protein, 112 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGPGTKLEIK

SEQ ID NO: 93 - DNA, 373 bp, synthetic construct; identical to SEQ ID NO: 69
  source 1..373: mol_type = other DNA

SEQ ID NO: 94 - protein, 124 aa, synthetic construct; identical to SEQ ID NO: 70

SEQ ID NO: 95 - DNA, 337 bp, synthetic construct
  source 1..337: mol_type = other DNA
gatattgtgatgactcagtctccactctccctgcccgtcacccctggagagccggcctccatctcctgcaggtctagtcagagcctcctgtatagtattggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacacttttggccaggggaccaagctggagatcaaac

SEQ ID NO: 96 - protein, 112 aa, synthetic construct
DIVMTQSPLSLPVTPGEPASISCRSSQSLLYSIGYNYLDWYLQKPGQSPQLLIYLGSNRASGVPDRFSGSGSGTDFTLKISRVEAEDVGVYYCMQALQTPYTFGQGTKLEIK

SEQ ID NO: 97 - DNA, 375 bp, synthetic construct
  source 1..375: mol_type = other DNA
caggtgcagctggtggagtctgagggactcttggaacagcctggggggtccctgagactctcctgtgcagcctctggattcaactttagagactttgccatgacctgggtccgccaggctccagggaaggggctggagtgggtctcatctattagtggtagtggtagtaatacatactacgcagactccgtgaagggccggttcaccatctccagagacaattccaaccacacgctgtatctgcgaatgaacagcctgagagccgaagacacggccgtgtattactgtgcgaaagatcgactctctataacaattcgcccacgctattacggtctggacgtctggggccaagggtccacggtcaccgtctcctca

SEQ ID NO: 98 - protein, 125 aa, synthetic construct
QVQLVESEGLLEQPGGSLRLSCAASGFNFRDFAMTWVRQAPGKGLEWVSSISGSGSNTYYADSVKGRFTISRDNSNHTLYLRMNSLRAEDTAVYYCAKDRLSITIRPRYYGLDVWGQGSTVTVSS

SEQ ID NO: 99 - DNA, 24 bp, synthetic construct
  source 1..24: mol_type = other DNA
ggattcaactttagagactttgcc

SEQ ID NO: 100 - protein, 8 aa, synthetic construct
GFNFRDFA

SEQ ID NO: 101 - DNA, 24 bp, synthetic construct
  source 1..24: mol_type = other DNA
attagtggtagtggtagtaataca

SEQ ID NO: 102 - protein, 8 aa, synthetic construct
ISGSGSNT

SEQ ID NO: 103 - DNA, 54 bp, synthetic construct
  source 1..54: mol_type = other DNA
gcgaaagatcgactctctataacaattcgcccacgctattacggtctggacgtc

SEQ ID NO: 104 - protein, 18 aa, synthetic construct
AKDRLSITIRPRYYGLDV

SEQ ID NO: 105 - DNA, 324 bp, synthetic construct
  source 1..324: mol_type = other DNA
gacatccagatgacccagtctccatcctccctgtctgcatctgtaggagacagagtcaccatcacttgccgggcgagtcaggacattagcaattattttgcctggtatcagcagaagccagggaaagttcctaagctcctgatctttgctgcatccactttgcatccaggggtcccatctcggttcagtggcagtggatctgggacagatttcactctcaccattcgcagcctgcagcctgaagatgttgcaacttattactgtcaaaaatatgacagtgccccgtacacttttggccaggggaccaaggtggaaatcaaacga

SEQ ID NO: 106 - protein, 108 aa, synthetic construct
DIQMTQSPSSLSASVGDRVTITCRASQDISNYFAWYQQKPGKVPKLLIFAASTLHPGVPSRFSGSGSGTDFTLTIRSLQPEDVATYYCQKYDSAPYTFGQGTKVEIKR

SEQ ID NO: 107 - DNA, 18 bp, synthetic construct
  source 1..18: mol_type = other DNA
caggacattagcaattat

SEQ ID NO: 108 - protein, 6 aa, synthetic construct
QDISNY

SEQ ID NO: 109-110 - skipped (2 entries)

SEQ ID NO: 111 - DNA, 27 bp, synthetic construct
  source 1..27: mol_type = other DNA
caaaaatatgacagtgccccgtacact

SEQ ID NO: 112 - protein, 9 aa, synthetic construct
QKYDSAPYT

SEQ ID NO: 113 - DNA, 372 bp, synthetic construct
  source 1..372: mol_type = other DNA
gaggtgcagctggtggagtctgagggactcttggaacagcctggggggtccctgagactctcctgtgcagcctctggattcaactttagagactttgccatgacctgggtccgccaggctccagggaaggggctggagtgggtctcatctattagtggtagtggtagtaatacatactacgcagactccgtgaagggccggttcaccatctccagagacaattccaaccacacgctgtatctgcgaatgaacagcctgagagccgaagacacggccgtgtattactgtgcgaaagatcgactctctataacaattcgcccacgctattacggtctggacgtctggggccaagggaccacggtcaccgtctcc

SEQ ID NO: 114 - protein, 124 aa, synthetic construct
EVQLVESEGLLEQPGGSLRLSCAASGFNFRDFAMTWVRQAPGKGLEWVSSISGSGSNTYYADSVKGRFTISRDNSNHTLYLRMNSLRAEDTAVYYCAKDRLSITIRPRYYGLDVWGQGTTVTVS

SEQ ID NO: 115 - DNA, 321 bp, synthetic construct
  source 1..321: mol_type = other DNA
gacatccagatgacccagtctccatcctccctgtctgcatctgtaggagacagagtcaccatcacttgccgggcgagtcaggacattagcaattattttgcctggtatcagcagaagccagggaaagttcctaagctcctgatctttgctgcatccactttgcatccaggggtcccatctcggttcagtggcagtggatctgggacagatttcactctcaccattcgcagcctgcagcctgaagatgttgcaacttattactgtcaaaaatatgacagtgccccgtacacttttggccaggggaccaagctggagatcaaa

SEQ ID NO: 116 - protein, 107 aa, synthetic construct
DIQMTQSPSSLSASVGDRVTITCRASQDISNYFAWYQQKPGKVPKLLIFAASTLHPGVPSRFSGSGSGTDFTLTIRSLQPEDVATYYCQKYDSAPYTFGQGTKLEIK

SEQ ID NO: 117 - DNA, 373 bp, synthetic construct
  source 1..373: mol_type = other DNA
gaggtgcagctggtggagtctgggggaggcttggtacagcctggggggtccctgagactctcctgtgcagcctctggattcaactttagagactttgccatgagctgggtccgccaggctccagggaaggggctggagtgggtctcagctattagtggtagtggtagtaatacatactacgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattacggtctggacgtctggggccaagggaccacggtcaccgtctcct

SEQ ID NO: 118 - protein, 124 aa, synthetic construct
EVQLVESGGGLVQPGGSLRLSCAASGFNFRDFAMSWVRQAPGKGLEWVSAISGSGSNTYYADSVKGRFTISRDNSKNTLYLQMNSLRAEDTAVYYCAKDRLSITIRPRYYGLDVWGQGTTVTVS

SEQ ID NO: 119 - DNA, 322 bp, synthetic construct
  source 1..322: mol_type = other DNA
gacatccagatgacccagtctccatcctccctgtctgcatctgtaggagacagagtcaccatcacttgccgggcgagtcaggacattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaaatatgacagtgccccgtacacttttggccaggggaccaagctggagatcaaac

SEQ ID NO: 120 - protein, 107 aa, synthetic construct
DIQMTQSPSSLSASVGDRVTITCRASQDISNYLAWYQQKPGKVPKLLIYAASTLQSGVPSRFSGSGSGTDFTLTISSLQPEDVATYYCQKYDSAPYTFGQGTKLEIK

SEQ ID NO: 121 - DNA, 357 bp, synthetic construct
  source 1..357: mol_type = other DNA
caggtgcagctggtggagtctgggggaggcttggtacagcctggggggtccctgagactctcctgtgtagcttctggattcacccttaacaactttgtcatgaactgggtccgccaggttccagggaagggactggagtgggtctcttttattagtgctagtggtggtagtatatactacgcagactccgtgaagggccggttcaccatctccagagacacttccaagaacacattatatctgcaaatgaacagcctgagagccgacgacacggccgtctattactgtgcgaaatccccgtataactggaacccctttgactattggggccagggaaccacggtcaccgtctcctca

SEQ ID NO: 122 - protein, 119 aa, synthetic construct
QVQLVESGGGLVQPGGSLRLSCVASGFTLNNFVMNWVRQVPGKGLEWVSFISASGGSIYYADSVKGRFTISRDTSKNTLYLQMNSLRADDTAVYYCAKSPYNWNPFDYWGQGTTVTVSS

SEQ ID NO: 123 - DNA, 24 bp, synthetic construct
  source 1..24: mol_type = other DNA
ggattcacccttaacaactttgtc

SEQ ID NO: 124 - protein, 8 aa, synthetic construct
GFTLNNFV

SEQ ID NO: 125 - DNA, 24 bp, synthetic construct
  source 1..24: mol_type = other DNA
attagtgctagtggtggtagtata

SEQ ID NO: 126 - protein, 8 aa, synthetic construct
ISASGGSI

SEQ ID NO: 127 - DNA, 36 bp, synthetic construct
  source 1..36: mol_type = other DNA
gcgaaatccccgtataactggaacccctttgactat

SEQ ID NO: 128 - protein, 12 aa, synthetic construct
AKSPYNWNPFDY

SEQ ID NO: 129 - DNA, 327 bp, synthetic construct
  source 1..327: mol_type = other DNA
gacatccagttgacccagtctccagccaccctgtctgtgtctccaggggaacgagccaccctctcctgcagggccagtctgagtgttagcagcaaattagcctggtaccagcagacacctggccaggctcccagactcctcatctatagtgcctccacccgggccactggtatcccagtcaggttcagtggcagtgggtctgggacagagttcactctcaccatcagcagcctgcagtctgaagattttgcggtttattactgtcagcagtataatcattggcctccgtacacttttggccaggggaccaaggtggagatcaaacga

SEQ ID NO: 130 - protein, 109 aa, synthetic construct
DIQLTQSPATLSVSPGERATLSCRASLSVSSKLAWYQQTPGQAPRLLIYSASTRATGIPVRFSGSGSGTEFTLTISSLQSEDFAVYYCQQYNHWPPYTFGQGTKVEIKR

SEQ ID NO: 131 - DNA, 18 bp, synthetic construct
  source 1..18: mol_type = other DNA
ctgagtgttagcagcaaa

SEQ ID NO: 132 - protein, 6 aa, synthetic construct
LSVSSK

SEQ ID NO: 133-134 - skipped (2 entries)

SEQ ID NO: 135 - DNA, 30 bp, synthetic construct
  source 1..30: mol_type = other DNA
cagcagtataatcattggcctccgtacact

SEQ ID NO: 136 - protein, 10 aa, synthetic construct
QQYNHWPPYT

SEQ ID NO: 137 - DNA, 357 bp, synthetic construct
  source 1..357: mol_type = other DNA
gaggtgcagctggtggagtctgggggaggcttggtacagcctggggggtccctgagactctcctgtgtagcttctggattcacccttaacaactttgtcatgaactgggtccgccaggttccagggaagggactggagtgggtctcttttattagtgctagtggtggtagtatatactacgcagactccgtgaagggccggttcaccatctccagagacacttccaagaacacattatatctgcaaatgaacagcctgagagccgacgacacggccgtctattactgtgcgaaatccccgtataactggaacccctttgactattggggccagggaaccctggtcaccgtctcctca

SEQ ID NO: 138 - protein, 119 aa, synthetic construct
EVQLVESGGGLVQPGGSLRLSCVASGFTLNNFVMNWVRQVPGKGLEWVSFISASGGSIYYADSVKGRFTISRDTSKNTLYLQMNSLRADDTAVYYCAKSPYNWNPFDYWGQGTLVTVSS

SEQ ID NO: 139 - DNA, 324 bp, synthetic construct
  source 1..324: mol_type = other DNA
gaaatagtgatgacgcagtctccagccaccctgtctgtgtctccaggggaacgagccaccctctcctgcagggccagtctgagtgttagcagcaaattagcctggtaccagcagacacctggccaggctcccagactcctcatctatagtgcctccacccgggccactggtatcccagtcaggttcagtggcagtgggtctgggacagagttcactctcaccatcagcagcctgcagtctgaagattttgcggtttattactgtcagcagtataatcattggcctccgtacacttttggccaggggaccaagctggagatcaaa

SEQ ID NO: 140 - protein, 108 aa, synthetic construct
EIVMTQSPATLSVSPGERATLSCRASLSVSSKLAWYQQTPGQAPRLLIYSASTRATGIPVRFSGSGSGTEFTLTISSLQSEDFAVYYCQQYNHWPPYTFGQGTKLEIK

SEQ ID NO: 141 - DNA, 357 bp, synthetic construct
  source 1..357: mol_type = other DNA
gaggtgcagctggtggagtctgggggaggcttggtacagcctggggggtccctgagactctcctgtgcagcctctggattcacccttaacaactttgtcatgagctgggtccgccaggctccagggaaggggctggagtgggtctcagctattagtgctagtggtggtagtatatactacgcagactccgtgaagggccggttcaccatctccagagacaattccaagaacacgctgtatctgcaaatgaacagcctgagagccgaggacacggccgtatattactgtgcgaaatccccgtataactggaacccctttgactattggggccagggaaccctggtcaccgtctcctca

SEQ ID NO: 142 - protein, 119 aa, synthetic construct
EVQLVESGGGLVQPGGSLRLSCAASGFTLNNFVMSWVRQAPGKGLEWVSAISASGGSIYYADSVKGRFTISRDNSKNTLYLQMNSLRAEDTAVYYCAKSPYNWNPFDYWGQGTLVTVSS

SEQ ID NO: 143 - DNA, 325 bp, synthetic construct
  source 1..325: mol_type = other DNA
gaaatagtgatgacgcagtctccagccaccctgtctgtgtctccaggggaaagagccaccctctcctgcagggccagtctgagtgttagcagcaaattagcctggtaccagcagaaacctggccaggctcccaggctcctcatctatagtgcctccaccagggccactggtatcccagccaggttcagtggcagtgggtctgggacagagttcactctcaccatcagcagcctgcagtctgaagattttgcagtttattactgtcagcagtataatcattggcctccgtacacttttggccaggggaccaagctggagatcaaac

SEQ ID NO: 144 - protein, 108 aa, synthetic construct
EIVMTQSPATLSVSPGERATLSCRASLSVSSKLAWYQQKPGQAPRLLIYSASTRATGIPARFSGSGSGTEFTLTISSLQSEDFAVYYCQQYNHWPPYTFGQGTKLEIK

SEQ ID NO: 145 - DNA, 375 bp, synthetic construct
  source 1..375: mol_type = other DNA
caggtgcagctggtggagtctgggggaggcttggaacagccgggggggtccctgagactctcctgtgcaggctctggattcacctttagagactatgccatgacctgggtccgccaggctccagggaagggactggagtgggtctcatctattagtggttccggtggtaacacatactacgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggtccacggtcaccgtctcctca

SEQ ID NO: 146 - protein, 125 aa, synthetic construct
QVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGSTVTVSS

SEQ ID NO: 147 - DNA, 24 bp, synthetic construct
  source 1..24: mol_type = other DNA
ggattcacctttagagactatgcc

SEQ ID NO: 148 - protein, 8 aa, synthetic construct
GFTFRDYA

SEQ ID NO: 149 - DNA, 24 bp, synthetic construct
  source 1..24: mol_type = other DNA
attagtggttccggtggtaacaca

SEQ ID NO: 150 - protein, 8 aa, synthetic construct
ISGSGGNT

SEQ ID NO: 151 - DNA, 54 bp, synthetic construct; identical to SEQ ID NO: 55
  source 1..54: mol_type = other DNA

SEQ ID NO: 152 - protein, 18 aa, synthetic construct
AKDRLSITIRPRYYGLDV

SEQ ID NO: 153 - DNA, 339 bp, synthetic construct
  source 1..339: mol_type = other DNA
gacatcgtgttgacccagtctccactctccctgcccgtcacccctggagagccggcctccatctcctgcaggtctagtcagagcctcctgtatagtattggatacaactatttggattggtacctgcagaagtcagggcagtctccacagctccttatctatttgggttctaatcgggcctccggggtccctgacaggttcagtggcagtggatcaggcacagattttacactgaaaatcagcagagtggaggctgaggatgttgggttttattactgcatgcaagctctacaaactccgtacacttttggccaggggaccaagctggagatcaaacga

SEQ ID NO: 154 - protein, 113 aa, synthetic construct
DIVLTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKR

SEQ ID NO: 155 - DNA, 33 bp, synthetic construct; identical to SEQ ID NO: 83
  source 1..33: mol_type = other DNA

SEQ ID NO: 156 - protein, 11 aa, synthetic construct
QSLLYSIGYNY

SEQ ID NO: 157-158 - skipped (2 entries)

SEQ ID NO: 159 - DNA, 27 bp, synthetic construct
  source 1..27: mol_type = other DNA
atgcaagctctacaaactccgtacact

SEQ ID NO: 160 - protein, 9 aa, synthetic construct
MQALQTPYT

SEQ ID NO: 161 - DNA, 372 bp, synthetic construct
  source 1..372: mol_type = other DNA
gaggtgcagctggtggagtctgggggaggcttggaacagccgggggggtccctgagactctcctgtgcaggctctggattcacctttagagactatgccatgacctgggtccgccaggctccagggaagggactggagtgggtctcatctattagtggttccggtggtaacacatactacgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggaccacggtcaccgtctcc

SEQ ID NO: 162 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 163 - DNA, 336 bp, synthetic construct
  source 1..336: mol_type = other DNA
gacatcgtgatgacccagtctccactctccctgcccgtcacccctggagagccggcctccatctcctgcaggtctagtcagagcctcctgtatagtattggatacaactatttggattggtacctgcagaagtcagggcagtctccacagctccttatctatttgggttctaatcgggcctccggggtccctgacaggttcagtggcagtggatcaggcacagattttacactgaaaatcagcagagtggaggctgaggatgttgggttttattactgcatgcaagctctacaaactccgtacacttttggccaggggaccaagctggagatcaaa

SEQ ID NO: 164 - protein, 112 aa, synthetic construct [sequence omitted]

SEQ ID NO: 165 - DNA, 373 bp, synthetic construct
  source 1..373: mol_type = other DNA
gaggtgcagctggtggagtctgggggaggcttggtacagcctggggggtccctgagactctcctgtgcagcctctggattcacctttagagactatgccatgagctgggtccgccaggctccagggaaggggctggagtgggtctcagctattagtggttccggtggtaacacatactacgcagactccgtgaagggccggttcaccatctccagagacaattccaagaacacgctgtatctgcaaatgaacagcctgagagccgaggacacggccgtatattactgtgcgaaagatcgactctctataacaattcgcccacgctattatggtttggacgtctggggccaagggaccacggtcaccgtctcct

SEQ ID NO: 166 - protein, 124 aa, synthetic construct; identical to SEQ ID NO: 70

SEQ ID NO: 167 - DNA, 337 bp, synthetic construct; identical to SEQ ID NO: 95
  source 1..337: mol_type = other DNA

SEQ ID NO: 168 - protein, 112 aa, synthetic construct; identical to SEQ ID NO: 96

SEQ ID NO: 169 - DNA, 375 bp, synthetic construct
  source 1..375: mol_type = other DNA
caggtgcagctggtggagtctgggggagtcttggagcagcctggggggtccctgagactctcctgtacagcctctggattcacctttagagactatgccatgacctgggtccgccaggctccagggaaggggctggagtgggtctcatctattagtggtagtggtggtaatacatactacgcagactccgtgaggggccggttcaccatctccagagacaactccaaccacacgctgtatctgcaaatgaacagcctgagagccgaagacacggccgtatattactgtgcgaaagatcgactctccataacaattcgcccacgctattacggtttggacgtctggggccaagggtccacggtcaccgtctcctca

SEQ ID NO: 170 - protein, 125 aa, synthetic construct
QVQLVESGGVLEQPGGSLRLSCTASGFTFRDYAMTWVRQAPGKGLEWVSSISGSGGNTYYADSVRGRFTISRDNSNHTLYLQMNSLRAEDTAVYYCAKDRLSITIRPRYYGLDVWGQGSTVTVSS

SEQ ID NO: 171 - DNA, 24 bp, synthetic construct
  source 1..24: mol_type = other DNA
ggattcacctttagagactatgcc

SEQ ID NO: 172 - protein, 8 aa, synthetic construct
GFTFRDYA

SEQ ID NO: 173 - DNA, 24 bp, synthetic construct
  source 1..24: mol_type = other DNA
attagtggtagtggtggtaataca

SEQ ID NO: 174 - protein, 8 aa, synthetic construct
ISGSGGNT

SEQ ID NO: 175 - DNA, 54 bp, synthetic construct
  source 1..54: mol_type = other DNA
gcgaaagatcgactctccataacaattcgcccacgctattacggtttggacgtc

SEQ ID NO: 176 - protein, 18 aa, synthetic construct
AKDRLSITIRPRYYGLDV

SEQ ID NO: 177 - DNA, 324 bp, synthetic construct
  source 1..324: mol_type = other DNA
gatattgtgatgacccagtctccatcctccctgtctgcatctgtaggagacagagtcaccattacttgccgggcgagtcaggacattagcaattattttgcctggtatcagcagaagccagggaaagttcctaaactcctgatctttgctgcatccactttgcatccaggggtcccatctcggttcagtggcagtggatctgggacagatttcactctcaccattagtagcctgcagcctgaagatgttgcaacttattactgtcaaaagtataacagtgccccgtacacttttggccaggggaccaaggtggaaatcaaacga

SEQ ID NO: 178 - protein, 108 aa, synthetic construct
DIVMTQSPSSLSASVGDRVTITCRASQDISNYFAWYQQKPGKVPKLLIFAASTLHPGVPSRFSGSGSGTDFTLTISSLQPEDVATYYCQKYNSAPYTFGQGTKVEIKR

SEQ ID NO: 179 - DNA, 18 bp, synthetic construct
  source 1..18: mol_type = other DNA
caggacattagcaattat

SEQ ID NO: 180 - protein, 6 aa, synthetic construct
QDISNY

SEQ ID NO: 181-182 - skipped (2 entries)

SEQ ID NO: 183 - DNA, 27 bp, synthetic construct
  source 1..27: mol_type = other DNA
caaaagtataacagtgccccgtacact

SEQ ID NO: 184 - protein, 9 aa, synthetic construct
QKYNSAPYT

SEQ ID NO: 185 - DNA, 372 bp, synthetic construct
  source 1..372: mol_type = other DNA
gaggtgcagctggtggagtctgggggagtcttggagcagcctggggggtccctgagactctcctgtacagcctctggattcacctttagagactatgccatgacctgggtccgccaggctccagggaaggggctggagtgggtctcatctattagtggtagtggtggtaatacatactacgcagactccgtgaggggccggttcaccatctccagagacaactccaaccacacgctgtatctgcaaatgaacagcctgagagccgaagacacggccgtatattactgtgcgaaagatcgactctccataacaattcgcccacgctattacggtttggacgtctggggccaagggaccacggtcaccgtctcc

SEQ ID NO: 186 - protein, 124 aa, synthetic construct
EVQLVESGGVLEQPGGSLRLSCTASGFTFRDYAMTWVRQAPGKGLEWVSSISGSGGNTYYADSVRGRFTISRDNSNHTLYLQMNSLRAEDTAVYYCAKDRLSITIRPRYYGLDVWGQGTTVTVS

SEQ ID NO: 187 - DNA, 321 bp, synthetic construct
  source 1..321: mol_type = other DNA
gacatccagatgacccagtctccatcctccctgtctgcatctgtaggagacagagtcaccattacttgccgggcgagtcaggacattagcaattattttgcctggtatcagcagaagccagggaaagttcctaaactcctgatctttgctgcatccactttgcatccaggggtcccatctcggttcagtggcagtggatctgggacagatttcactctcaccattagtagcctgcagcctgaagatgttgcaacttattactgtcaaaagtataacagtgccccgtacacttttggccaggggaccaagctggagatcaaa

SEQ ID NO: 188 - protein, 107 aa, synthetic construct
DIQMTQSPSSLSASVGDRVTITCRASQDISNYFAWYQQKPGKVPKLLIFAASTLHPGVPSRFSGSGSGTDFTLTISSLQPEDVATYYCQKYNSAPYTFGQGTKLEIK

SEQ ID NO: 189 - DNA, 373 bp, synthetic construct
  source 1..373: mol_type = other DNA
gaggtgcagctggtggagtctgggggaggcttggtacagcctggggggtccctgagactctcctgtgcagcctctggattcacctttagagactatgccatgagctgggtccgccaggctccagggaaggggctggagtgggtctcagctattagtggtagtggtggtaatacatactacgcagactccgtgaagggccggttcaccatctccagagacaattccaagaacacgctgtatctgcaaatgaacagcctgagagccgaggacacggccgtatattactgtgcgaaagatcgactctccataacaattcgcccacgctattacggtttggacgtctggggccaagggaccacggtcaccgtctcct

SEQ ID NO: 190 - protein, 124 aa, synthetic construct; identical to SEQ ID NO: 70

SEQ ID NO: 191 - DNA, 322 bp, synthetic construct
  source 1..322: mol_type = other DNA
gacatccagatgacccagtctccatcctccctgtctgcatctgtaggagacagagtcaccatcacttgccgggcgagtcaggacattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtacacttttggccaggggaccaagctggagatcaaac

SEQ ID NO: 192 - protein, 107 aa, synthetic construct
DIQMTQSPSSLSASVGDRVTITCRASQDISNYLAWYQQKPGKVPKLLIYAASTLQSGVPSRFSGSGSGTDFTLTISSLQPEDVATYYCQKYNSAPYTFGQGTKLEIK

SEQ ID NO: 193 - DNA, 355 bp, synthetic construct
  source 1..355: mol_type = other DNA
gaagtgcacctggtggaatctgggggaggcttggtacagcctggcaggtccctgagactctcctgtgaggcctctggattcacctttgatgattatgccatgcactgggtccggcaagctccggggaagggcctggaatgggtctcaggtcttagtcggacaagtgtcagtataggctatgcggactctgtgaagggccgattcaccatctccagagacaacgccaagaactccctttatttggaaatgaacagtctgagacctgaggacacggccttatattactgtgcaaaatgggggacccgggggtattttgactactggggccagggaaccctggtcaccgtctcctcag

SEQ ID NO: 194 - protein, 118 aa, synthetic construct
EVHLVESGGGLVQPGRSLRLSCEASGFTFDDYAMHWVRQAPGKGLEWVSGLSRTSVSIGYADSVKGRFTISRDNAKNSLYLEMNSLRPEDTALYYCAKWGTRGYFDYWGQGTLVTVSS

SEQ ID NO: 195 - DNA, 24 bp, synthetic construct
  source 1..24: mol_type = other DNA
ggattcacctttgatgattatgcc

SEQ ID NO: 196 - protein, 8 aa, synthetic construct
GFTFDDYA

SEQ ID NO: 197 - DNA, 24 bp, synthetic construct
  source 1..24: mol_type = other DNA
cttagtcggacaagtgtcagtata

SEQ ID NO: 198 - protein, 8 aa, synthetic construct
LSRTSVSI

SEQ ID NO: 199 - DNA, 33 bp, synthetic construct
  source 1..33: mol_type = other DNA
gcaaaatgggggacccgggggtattttgactac

SEQ ID NO: 200 - protein, 11 aa, synthetic construct
AKWGTRGYFDY

SEQ ID NO: 201 - DNA, 322 bp, synthetic construct
  source 1..322: mol_type = other DNA
gacatccagatgacccagtctccatcttccgtgtctgcatctgtgggagacagagtcaccatcacttgtcgggcgagtcaggatattagtatttggttagcctggtatcagcagagtccagggaaagcccctaaactcctgatcaatgttgcatcccgtttgcaaagtggggtcccatcaaggttcagcggcagtggatctgggacagatttcactctcaccatcaacagtctgcagcctgaagattttgtaacttactattgtcaacaggctaacagtttcccgatcaccttcggccaagggacacgactggcgaccaaac

SEQ ID NO: 202 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 203 - DNA, 18 bp, synthetic construct
  source 1..18: mol_type = other DNA
caggatattagtatttgg

SEQ ID NO: 204 - protein, 6 aa, synthetic construct
QDISIW

SEQ ID NO: 205-206 - skipped (2 entries)

SEQ ID NO: 207 - DNA, 27 bp, synthetic construct
  source 1..27: mol_type = other DNA
caacaggctaacagtttcccgatcacc

SEQ ID NO: 208 - protein, 9 aa, synthetic construct
QQANSFPIT

SEQ ID NO: 209 - DNA, 355 bp, synthetic construct
  source 1..355: mol_type = other DNA
gaggtgcagctggtggagtctgggggaggcttggtacagcctggcaggtccctgagactctcctgtgaggcctctggattcacctttgatgattatgccatgcactgggtccggcaagctccggggaagggcctggaatgggtctcaggtcttagtcggacaagtgtcagtataggctatgcggactctgtgaagggccgattcaccatctccagagacaacgccaagaactccctttatttggaaatgaacagtctgagacctgaggacacggccttatattactgtgcaaaatgggggacccgggggtattttgactactggggccagggaaccctggtcaccgtctcctcag

SEQ ID NO: 210 - protein, 118 aa, synthetic construct
EVQLVESGGGLVQPGRSLRLSCEASGFTFDDYAMHWVRQAPGKGLEWVSGLSRTSVSIGYADSVKGRFTISRDNAKNSLYLEMNSLRPEDTALYYCAKWGTRGYFDYWGQGTLVTVSS

SEQ ID NO: 211 - DNA, 322 bp, synthetic construct
  source 1..322: mol_type = other DNA
gacatccagatgacccagtctccatcttccgtgtctgcatctgtgggagacagagtcaccatcacttgtcgggcgagtcaggatattagtatttggttagcctggtatcagcagagtccagggaaagcccctaaactcctgatcaatgttgcatcccgtttgcaaagtggggtcccatcaaggttcagcggcagtggatctgggacagatttcactctcaccatcaacagtctgcagcctgaagattttgtaacttactattgtcaacaggctaacagtttcccgatcaccttcggccaagggacacgactggagattaaac

SEQ ID NO: 212 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLEIK

SEQ ID NO: 213 - DNA, 355 bp, synthetic construct
  source 1..355: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtcttagtcggacaagtgtcagtataggctatgcggactctgtgaagggccgattcaccatctccagagacaacgccaagaactccctgtatctgcaaatgaacagtctgagagctgaggacacggccttgtattactgtgcaaaatgggggacccgggggtattttgactactggggccaaggaaccctggtcaccgtctcctcag

SEQ ID NO: 214 - protein, 118 aa, synthetic construct
EVQLVESGGGLVQPGRSLRLSCAASGFTFDDYAMHWVRQAPGKGLEWVSGLSRTSVSIGYADSVKGRFTISRDNAKNSLYLQMNSLRAEDTALYYCAKWGTRGYFDYWGQGTLVTVSS

SEQ ID NO: 215 - DNA, 322 bp, synthetic construct
  source 1..322: mol_type = other DNA
gacatccagatgacccagtctccatcttccgtgtctgcatctgtaggagacagagtcaccatcacttgtcgggcgagtcaggatattagtatttggttagcctggtatcagcagaaaccagggaaagcccctaagctcctgatctatgttgcatccagtttgcaaagtggggtcccatcaaggttcagcggcagtggatctgggacagatttcactctcaccatcagcagcctgcagcctgaagattttgcaacttactattgtcaacaggctaacagtttcccgatcaccttcggccaagggacacgactggagattaaac

SEQ ID NO: 216 - protein, 107 aa, synthetic construct
DIQMTQSPSSVSASVGDRVTITCRASQDISIWLAWYQQKPGKAPKLLIYVASSLQSGVPSRFSGSGSGTDFTLTISSLQPEDFATYYCQQANSFPITFGQGTRLEIK

SEQ ID NO: 217 - DNA, 363 bp, synthetic construct
  source 1..363: mol_type = other DNA
gaggtgcagctgttggagtctgggggaggcttgctacagccgggggggtccctgagactctcctgtgcagcctctggaatcacctttagcacctatgccatgagctgggtccgtcaggctccagggagggggctggagtgggtctcagctattagtggtagtggtgatagcacatcctacgcagactccgtgaagggccggttcaccagctccagagacaattccaagaacacgctgtatctgcaaatgaacagcctgagagccgaggacacggccgtatattactgtgcgaaagtcatagcagctcgtcctcactggaacttcgatctctggggccgtggcaccctggtcactgtctcctca

SEQ ID NO: 218 - protein, 121 aa, synthetic construct
EVQLLESGGGLLQPGGSLRLSCAASGITFSTYAMSWVRQAPGRGLEWVSAISGSGDSTSYADSVKGRFTSSRDNSKNTLYLQMNSLRAEDTAVYYCAKVIAARPHWNFDLWGRGTLVTVSS

SEQ ID NO: 219 - DNA, 24 bp, synthetic construct
  source 1..24: mol_type = other DNA
ggaatcacctttagcacctatgcc

SEQ ID NO: 220 - protein, 8 aa, synthetic construct
GITFSTYA

SEQ ID NO: 221 - DNA, 24 bp, synthetic construct
  source 1..24: mol_type = other DNA
attagtggtagtggtgatagcaca

SEQ ID NO: 222 - protein, 8 aa, synthetic construct
ISGSGDST

SEQ ID NO: 223 - DNA, 42 bp, synthetic construct
  source 1..42: mol_type = other DNA
gcgaaagtcatagcagctcgtcctcactggaacttcgatctc

SEQ ID NO: 224 - protein, 14 aa, synthetic construct
AKVIAARPHWNFDL

SEQ ID NO: 225 - DNA, 324 bp, synthetic construct
  source 1..324: mol_type = other DNA
gaaattgtgttgacacagtctccagccaccctgtctttgtctccaggggaaagagccaccctctcctgcagggccagtcagagtgttagtagatatttagcctggtatcaacagaaacctggccaggctcccaggctcctcatctatgatgcatccaacagggccactggcatcccagccaggttcagtggcagtgggtctgggacagacttcactctcaccatcagcagcctagagcctgaagattttggagtttattactgtcagcagcgtagtgactggccgctcactttcggcggagggaccaaggtggagatcaaacgg

SEQ ID NO: 226 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFGVYYCQQRSDWPLTFGGGTKVEIK

SEQ ID NO: 227 - DNA, 18 bp, synthetic construct
  source 1..18: mol_type = other DNA
cagagtgttagtagatat

SEQ ID NO: 228 - protein, 6 aa, synthetic construct
QSVSRY

SEQ ID NO: 229-230 - skipped (2 entries)

SEQ ID NO: 231 - DNA, 27 bp, synthetic construct
  source 1..27: mol_type = other DNA
cagcagcgtagtgactggccgctcact

SEQ ID NO: 232 - protein, 9 aa, synthetic construct
QQRSDWPLT

SEQ ID NO: 233 - DNA, 363 bp, synthetic construct; identical to SEQ ID NO: 217
  source 1..363: mol_type = other DNA

SEQ ID NO: 234 - protein, 121 aa, synthetic construct; identical to SEQ ID NO: 218

SEQ ID NO: 235 - DNA, 324 bp, synthetic construct; identical to SEQ ID NO: 225
  source 1..324: mol_type = other DNA

SEQ ID NO: 236 - protein, 107 aa, synthetic construct; identical to SEQ ID NO: 226

SEQ ID NO: 237 - DNA, 363 bp, synthetic construct
  source 1..363: mol_type = other DNA
gaggtgcagctgttggagtctgggggaggcttggtacagccgggggggtccctgagactctcctgtgcagcctctggaatcacctttagcacctatgccatgagctgggtccgtcaggctccagggaaggggctggagtgggtctcagctattagtggtagtggtgatagcacatactacgcagactccgtgaagggccggttcaccatctccagagacaattccaagaacacgctgtatctgcaaatgaacagcctgagagccgaggacacggccgtatattactgtgcgaaagtcatagcagctcgtcctcactggaacttcgatctctggggccgtggcaccctggtcactgtctcctca

SEQ ID NO: 238 - protein, 121 aa, synthetic construct
EVQLLESGGGLVQPGGSLRLSCAASGITFSTYAMSWVRQAPGKGLEWVSAISGSGDSTYYADSVKGRFTISRDNSKNTLYLQMNSLRAEDTAVYYCAKVIAARPHWNFDLWGRGTLVTVSS

SEQ ID NO: 239 - DNA, 324 bp, synthetic construct
  source 1..324: mol_type = other DNA
gaaattgtgttgacacagtctccagccaccctgtctttgtctccaggggaaagagccaccctctcctgcagggccagtcagagtgttagtagatatttagcctggtatcaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagtgactggccgctcactttcggcggagggaccaaggtggagatcaaacgg

SEQ ID NO: 240 - protein, 108 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSDWPLTFGGGTKVEIKR

SEQ ID NO: 241 - DNA, 366 bp, synthetic construct
  source 1..366: mol_type = other DNA
caggtgcagctggtggagtctgggggaggcgtggtccagcctgggaggtccctgagactcacctgtgcagcctctggattcaccttcagtagtaatggcatgcactgggtccgccaggctccaggcaaggggctggagtgggtggcaattatatcatatgatggaaataatcaatactatgcagactccgtgaagggccgattcaccatctccagagacaattccaagcacacgctgtatctggaaatgaacagcctgagagctgaggacacggctgtgtattactgtacaaaagccatctctataagtggaacttacaactggttcgattcctggggccagggaaccctggtcaccgtctcctca

SEQ ID NO: 242 - protein, 122 aa, synthetic construct
QVQLVESGGGVVQPGRSLRLTCAASGFTFSSNGMHWVRQAPGKGLEWVAIISYDGNNQYYADSVKGRFTISRDNSKHTLYLEMNSLRAEDTAVYYCTKAISISGTYNWFDSWGQGTLVTVSS

SEQ ID NO: 243 - DNA, 24 bp, synthetic construct
  source 1..24: mol_type = other DNA
ggattcaccttcagtagtaatggc

SEQ ID NO: 244 - protein, 8 aa, synthetic construct
GFTFSSNG

SEQ ID NO: 245 - DNA, 24 bp, synthetic construct
  source 1..24: mol_type = other DNA
atatcatatgatggaaataatcaa

SEQ ID NO: 246 - protein, 8 aa, synthetic construct
ISYDGNNQ

SEQ ID NO: 247 - DNA, 45 bp, synthetic construct
  source 1..45: mol_type = other DNA
acaaaagccatctctataagtggaacttacaactggttcgattcc

SEQ ID NO: 248 - protein, 15 aa, synthetic construct
TKAISISGTYNWFDS

SEQ ID NO: 249 - DNA, 324 bp, synthetic construct
  source 1..324: mol_type = other DNA
gaaattgtattgacacagtctccagccatcctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcaacagcgtagcaactggccgctcactttcggcggagggaccaaggtggagatcaaacgg

SEQ ID NO: 250 - protein, 107 aa, synthetic construct
EIVLTQSPAILSLSPGERATLSCRASQSVSRYLAWYQQKPGQAPRLLIYDASNRATGIPARFSGSGSGTDFTLTISSLEPEDFAVYYCQQRSNWPLTFGGGTKVEIK

SEQ ID NO: 251 - DNA, 18 bp, synthetic construct
  source 1..18: mol_type = other DNA
cagagtgttagcaggtac

SEQ ID NO: 252 - protein, 6 aa, synthetic construct
QSVSRY

SEQ ID NO: 253-254 - skipped (2 entries)

SEQ ID NO: 255 - DNA, 27 bp, synthetic construct
  source 1..27: mol_type = other DNA
caacagcgtagcaactggccgctcact

SEQ ID NO: 256 - protein, 9 aa, synthetic construct
QQRSNWPLT

SEQ ID NO: 257 - DNA, 366 bp, synthetic construct; identical to SEQ ID NO: 241
  source 1..366: mol_type = other DNA

SEQ ID NO: 258 - protein, 122 aa, synthetic construct; identical to SEQ ID NO: 242

SEQ ID NO: 259 - DNA, 324 bp, synthetic construct; identical to SEQ ID NO: 249
  source 1..324: mol_type = other DNA

SEQ ID NO: 260 - protein, 107 aa, synthetic construct; identical to SEQ ID NO: 250

SEQ ID NO: 261 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagtaatggcatgcactgggtccgccaggctccaggcaaggggctggagtgggtggcagttatatcatatgatggaaataatcaatactatgcagactccgtgaagggccgattcaccatctccagagacaattccaagaacacgctgtatctgcaaatgaacagcctgagagctgaggacacggctgtgtattactgtacaaaagccatctctataagtggaacttacaactggttcgattcctggggccagggaaccctggtcaccgtctcctca

SEQ ID NO: 262 - protein, 122 aa, synthetic construct
QVQLVESGGGVVQPGRSLRLSCAASGFTFSSNGMHWVRQAPGKGLEWVAVISYDGNNQYYADSVKGRFTISRDNSKNTLYLQMNSLRAEDTAVYYCTKAISISGTYNWFDSWGQGTLVTVSS

SEQ ID NO: 263 - DNA, 324 bp, synthetic construct
  source 1..324: mol_type = other DNA
gaaattgtattgacacagtctccagccaccctgtctttgtctccaggggaaagagccaccctctcctgcagggccagtcagagtgttagcaggtacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcaacagcgtagcaactggccgctcactttcggcggagggaccaaggtggagatcaaacgg

SEQ ID NO: 264 - protein, 108 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQRSNWPLTFGGGTKVEIKR

SEQ ID NO: 265-270 - skipped (6 entries)

SEQ ID NO: 271 - protein, 330 aa, synthetic construct [sequence omitted]

SEQ ID NO: 272 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 273 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 274 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 275 - protein, 231 aa, Macaca fascicularis
MGWLCSGLLFPVSCLVLLQVASSGSMKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQ